(19) United States
(12) Patent Application Publication
(10) Pub. No.: US 2025/0034632 A1
(43) Pub. Date: Jan. 30, 2025

(54) NUCLEIC ACID ANALYSIS SYSTEM

(71) Applicant: SONY GROUP CORPORATION, TOKYO (JP)

(72) Inventors: KENSUKE KOJIMA, TOKYO (JP); YUSAKU NAKASHIMA, TOKYO (JP); MASAYOSHI AKITA, TOKYO (JP); RYOUSUKE MINAMI, TOKYO (JP)

(21) Appl. No.: 18/559,835

(22) PCT Filed: Feb. 15, 2022
(86) PCT No.: PCT/JP2022/005951

§ 371 Date: Nov. 9, 2023

(30) Foreign Application Priority Data
May 21, 2021 (JP) 2021-086006

(51) Int. Cl. C12Q 1/6851; B01L 7/00

(52) U.S. Cl. CPC C12Q 1/6851; B01L 7/52; B01L 2200/14

(57) ABSTRACT

An object of the present disclosure is to improve test accuracy of a nucleic acid detection method.

The present disclosure provides a nucleic acid analysis system including: an amplification reaction execution unit that executes a nucleic acid amplification reaction on a biological sample; and a control unit that controls the amplification reaction execution unit, the control unit being configured to determine whether to adjust a nucleic acid amplification reaction on the basis of a detection result of the nucleic acid amplification reaction in one or more positive controls, and to generate a determination result for the biological sample on the basis of the detection result of the nucleic acid amplification reaction in the one or more positive controls and a detection result of the nucleic acid amplification reaction in the biological sample.

### TECHNICAL FIELD

The present disclosure relates to a nucleic acid analysis system.

### BACKGROUND ART

A nucleic acid amplification process is performed to analyze a nucleic acid contained in a biological sample. The nucleic acid amplification process is sometimes performed to test whether being infected with a pathogen such as a virus or a bacterium. For example, whether a nucleic acid derived from a pathogen is contained in a biological sample such as a swab of a nasal cavity or a pharynx or saliva is determined by detecting a fluorescence signal generated in the nucleic acid amplification process.

As a device used for the nucleic acid amplification process, for example, Patent Document 1 below discloses a microchip in which a region into which a solution is introduced is disposed such that the inside thereof has a pressure negative to atmospheric pressure. Patent Document 2 below discloses a microchip including a plurality of substrate layers and bonding layers provided at boundary surfaces between the substrate layers and configured to include a silicon compound, in which at least one of the bonding layers is configured to include an organic silicon compound.

Furthermore, as a nucleic acid analysis technique, for example, Patent Document 3 below discloses a base sequence analysis method including a nucleic acid amplification procedure of obtaining an amplification product by a nucleic acid amplification reaction, a turbidity measurement procedure of measuring turbidity of a reaction solution of the nucleic acid amplification reaction, and a melting curve analysis procedure of performing melting curve analysis of a probe nucleic acid chain and the amplification product at a reaction site of the nucleic acid amplification reaction.

### CITATION LIST

### Patent Document

Patent Document 1: Japanese Patent Application Laid-Open No. 2011-163984
Patent Document 2: WO 2014/010299
Patent Document 3: Japanese Patent Application Laid-Open No. 2014-082987

### SUMMARY OF THE INVENTION

### Problems to be Solved by the Invention

Nucleic acid detection methods of performing the nucleic acid amplification process are used for detecting pathogen viruses such as a novel coronavirus (SARS-CoV2) and an influenza virus. Currently used nucleic acid detection methods have achieved a certain level of test accuracy, but there are still cases where an erroneous determination is made. In particular, in a case where the number of copies of a target nucleic acid contained in a specimen is small or in a case where a large amount of impurities is contained in the specimen, an erroneous determination may be made. In this regard, an object of the present disclosure is to improve test accuracy of a nucleic acid detection method.

### Solutions to Problems

The present inventors have found that the test accuracy of the nucleic acid detection method can be improved by a nucleic acid analysis system having a specific configuration.

That is, the present disclosure provides
a nucleic acid analysis system including:
an amplification reaction execution unit that executes a nucleic acid amplification reaction on a biological sample; and
a control unit that controls the amplification reaction execution unit,
the control unit being configured to
determine whether to adjust a nucleic acid amplification reaction on the basis of a detection result of the nucleic acid amplification reaction in one or more positive controls, and
generate a determination result for the biological sample on the basis of the detection result of the nucleic acid amplification reaction in the one or more positive controls and a detection result of the nucleic acid amplification reaction in the biological sample.

The control unit may generate determination reference data on the basis of the detection result of the nucleic acid amplification reaction in the one or more positive controls, and then,
determine whether to adjust the nucleic acid amplification reaction on the basis of the determination reference data.

The control unit may determine whether to adjust the nucleic acid amplification reaction on the basis of the determination reference data and standard reference data.

The control unit may determine whether to adjust the nucleic acid amplification reaction on the basis of a Tt value or a Ct value of the nucleic acid amplification reaction in the one or more positive controls.

The control unit may determine whether to adjust the nucleic acid amplification reaction using a trained model.

The control unit may adjust a reaction time or the number of reaction cycles of the nucleic acid amplification reaction in adjustment of the nucleic acid amplification reaction.

The control unit may determine that the nucleic acid amplification reaction for the biological sample is invalid in a case where the determination reference data cannot be generated.

The control unit may execute a determination result generation process of generating a determination result for the biological sample in response to determining not to adjust the nucleic acid amplification reaction.

In the determination result generation process, the control unit may determine whether a target nucleic acid is detected in the biological sample.

In the determination result generation process, the control unit may further execute a wrong answer determination process of determining whether a determination result regarding whether the target nucleic acid has been detected in the biological sample is a wrong answer.

The control unit may execute the wrong answer determination process in a case where the detection result of the nucleic acid amplification reaction in the biological sample does not satisfy a predetermined condition.

The control unit may refer to a detection result of the nucleic acid amplification reaction in a negative sample in the wrong answer determination process.

The detection result of the nucleic acid amplification reaction in the negative sample may include a detection result of the nucleic acid amplification reaction executed on another sample determined to be negative.

The control unit may execute the wrong answer determination process using a trained model.

The control unit may output notification data regarding a false positive on the basis of a determination result in the wrong answer determination process.

The control unit may output a screen regarding whether to execute additional analysis for generating a determination result for the biological sample on the basis of the determination result in the wrong answer determination process.

The additional analysis may be melting curve analysis.

In one embodiment of the present disclosure, the nucleic acid analysis system may include:
a nucleic acid amplification device including the amplification reaction execution unit; and
an information processing device including the control unit.

In another embodiment of the present disclosure, the nucleic acid analysis system may include:
a nucleic acid amplification device including the amplification reaction execution unit; and
a server device including the control unit.

Furthermore, the present disclosure provides a nucleic acid analysis system including:
a control unit that controls a nucleic acid amplification reaction for a biological sample,
the control unit being configured to determine whether to adjust the nucleic acid amplification reaction on the basis of a detection result of a nucleic acid amplification reaction in one or more positive controls, and
to generate a determination result for the biological sample on the basis of the detection result of the nucleic acid amplification reaction in the one or more positive controls and a detection result of the nucleic acid amplification reaction in the biological sample.

### BRIEF DESCRIPTION OF DRAWINGS

FIG. 1A is a diagram illustrating a configuration example of a nucleic acid analysis system according to the present disclosure.

FIG. 1B is a diagram illustrating another configuration example of the nucleic acid analysis system according to the present disclosure.

FIG. 2 is a view illustrating a configuration example of a nucleic acid amplification device.

FIG. 3 is a diagram illustrating a configuration example of a control unit.

FIG. 4 is a schematic view of an example of a chip.

FIG. 5 is a view for describing well assignment.

FIG. 6 is an example of a flowchart of processing executed by the nucleic acid analysis system.

FIG. 7 is an example of a flowchart of a nucleic acid amplification reaction adjustment process.

FIG. 8 is an example of a flowchart of a determination process.

FIG. 9 is an example of a flowchart of a determination process.

FIG. 10 is an example of a flowchart of a determination process.

FIG. 11 is a view illustrating a modified example of the nucleic acid analysis system.

FIG. 12 is a view illustrating the modified example of the nucleic acid analysis system.

FIG. 13 is a schematic view for illustrating primers used in an LAMP method.

FIG. 14 is a schematic view for describing a reaction in the LAMP method.

FIG. 15 is a schematic view for describing the reaction in the LAMP method.

FIG. 16 is a view for describing primers used in PCR.

FIG. 17 is a view for describing a variation of a Tt value.

FIG. 18 is a view for describing a setting of a threshold.

FIG. 19 is a view for describing the setting of the threshold.

### MODE FOR CARRYING OUT THE INVENTION

Hereinafter, preferred modes for carrying out the present disclosure will be described. Note that embodiments described below illustrate representative embodiments of the present disclosure, and the scope of the present disclosure is not limited only to these embodiments. Note that the present disclosure will be described in the following order.
1. First embodiment (nucleic acid analysis system)
(1) Description of first embodiment
(2) Example of nucleic acid analysis system
(2-1) Amplification reaction execution unit
(2-2) Light source unit
(2-3) Detection unit
(2-4) Control unit
(2-5) Container
(2-5-1) Examples of primers used in LAMP method
(2-5-2) Examples of primers used in PCR
(3) Examples of processes executed by nucleic acid analysis system
(3-1) Nucleic acid amplification reaction adjustment process
(3-2) Determination process
(3-3) Modified example of determination process
(4) First modified example of nucleic acid analysis system
(5) Second modified example of nucleic acid analysis system

### 1. First Embodiment (Nucleic Acid Analysis System)

### (1) Description of First Embodiment

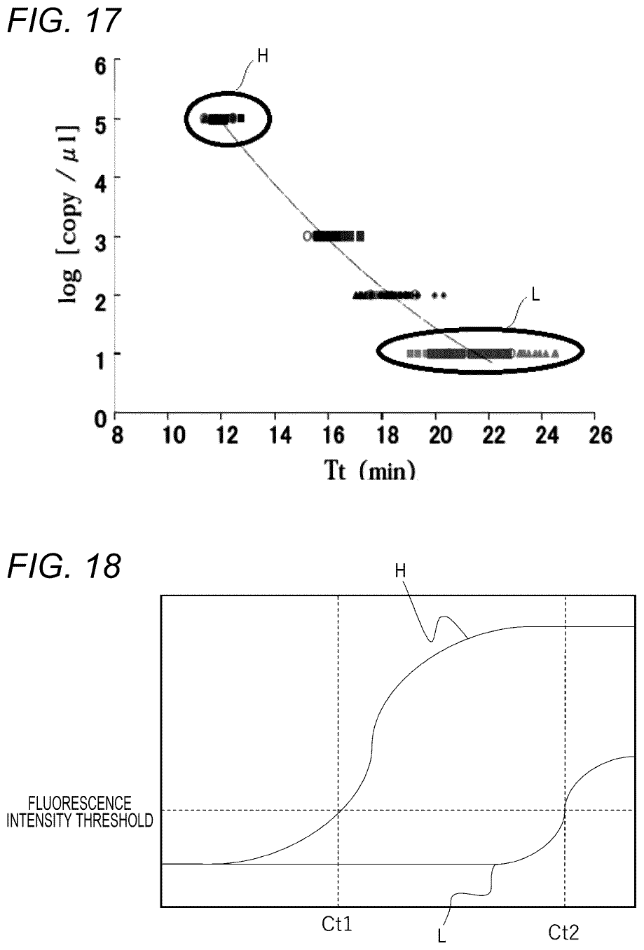

In conventional nucleic acid amplification tests, in a case where the amount of a target nucleic acid contained in a specimen (also referred to as a template amount or a template copy number) is small, a variation of a Ct value (also referred to as a threshold cycle or a Cq value) or a Tt value (also referred to as a threshold time) is likely to occur, and this variation also occurs in a PCR test having sufficiently high sensitivity. For example, as illustrated in FIG. 17, the lower the copy number, the larger the variation of the measured Tt value. For example, the variation of the Tt value is larger in a case where a template copy number indicated by reference sign L is small as compared with a case where a template copy number indicated by reference sign H is large. Therefore, in the above case, there is a high possibility that a determination result as to whether the target nucleic acid is present in the specimen will be a wrong answer (false negative or false positive).

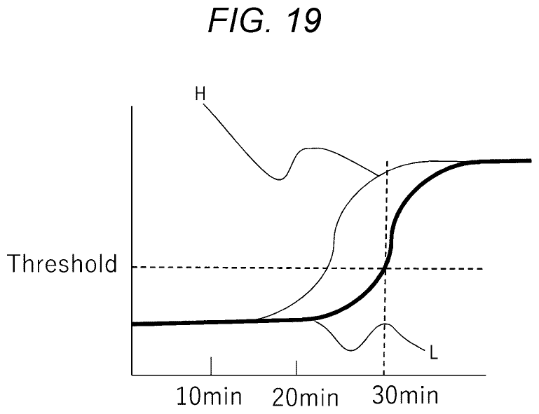

In order to keep the variation within a predetermined range, a threshold (threshold line) for making a positive determination (determining that the target nucleic acid is present in the specimen) is set to be high to some extent. In this case, however, the possibility of being determined as a false negative also increases. For example, regarding PCR, a case where an increase in fluorescence intensity is observed as the number of cycles increases in both the case of a high template copy number (indicated by reference sign H) and the case of a low copy number (reference sign L) is assumed as illustrated in FIG. 18. In this case, for example, in a case where Ct2 higher than Ct1 is adopted as the threshold, in the case of a low copy sample, the variation of the Ct value increases every test, and thus, a case in which the Ct value falls below Ct2 randomly occurs. In this manner, even the same specimen sample is determined to be negative in some cases or determined to be positive in other cases. Furthermore, this similarly applies to isothermal nucleic acid amplification. For example, as illustrated in FIG. 19, a nucleic acid amplification amount increases as a reaction time for amplification elapses in both the case of the high template copy number (indicated by reference sign H) and the case of the low copy number (reference sign L). In the case of the high copy number, an increase in the nucleic acid amplification amount can be confirmed at a lapse of 20 minutes, and the nucleic acid amplification amount exceeds the threshold at the lapse of 25 minutes. On the other hand, in the case of the low copy number, the nucleic acid amplification amount exceeds the threshold around 30 minutes because the variation increases every test. Therefore, in the case of the low copy number, even the same specimen sample is determined to be negative in some tests or determined to be positive in other tests.

In order to more reliably make a positive determination, it is desirable to set the Tt value to more than 30 minutes in consideration of the case of the low copy number, but tests are terminated with a standard test time in many cases. Therefore, copy number sensitivity generally in a range in which the variation is absorbed is set to a compensation range.

Furthermore, in the conventional nucleic acid amplification tests, there is also a problem that a nucleic acid amplification reaction inhibitory substance contained in a specimen brings about a matrix effect. In many nucleic acid amplification tests (for example, a PCR test, an isothermal nucleic acid amplification test, and the like), the specimen is pretreated in order to remove the inhibitory substance. However, since the inhibitory substance cannot be removed by 100%, a test result may be still affected by the matrix effect. The matrix effect delays a nucleic acid amplification reaction, that is, delays a fluorescence signal rise time, which brings about a variation of the Ct or Tt value.

If a variation of the Ct value is within 1.5, such a variation is allowed in many cases. However, for example, many nucleic acid amplification tests, actually conducted to determine the presence of a pathogen virus in a specimen, are conducted without knowing how much the matrix effect affects the test, particularly how much variation is caused in the Ct value.

In order to eliminate the influence of the matrix effect, it is conceivable to use a spike sample (internal control). For example, it is conceivable to prepare a plurality of (for example, three or more) samples for each specimen and add a known amount of a standard substance to one of the samples. However, it is necessary to handle a large number of specimens in a PCR test that is actually performed to determine the presence of a pathogen virus such as a novel coronavirus, so that batch processing is generally performed. Therefore, it is not practical to prepare a spike sample for each specimen. Furthermore, a variation due to dispensing work by a pipette may also occur in nucleic acid amplification tests being actually performed. The variation differs every test, and it is difficult to adopt such an accuracy control technique for coping with the variation in a nucleic acid amplification test in which batch processing is performed.

In view of such circumstances, for example, there is a case where copy number sensitivity that is about two orders of magnitude lower than a detection sensitivity limit (LoD) of a device to be used is adopted as a threshold for positive identification in a nucleic acid amplification test for a pathogen virus. In this case, the possibility of being determined to be negative increases even in a case where a minute amount of the pathogen virus is present in a specimen. Furthermore, in a case where the threshold is lowered, a false positive problem due to a signal of a non-specific reaction caused by a self-by-product of primers may also occur.

In this manner, the variation of the Ct value due to the low template copy number and a fluctuation in the fluorescence signal rise time due to the matrix effect can bring about a fluctuation in a positive or negative determination in the vicinity of a cut-off value in clinical tests.

A nucleic acid analysis system according to the present disclosure includes a control unit configured to determine whether to adjust the nucleic acid amplification reaction on the basis of a detection result of a nucleic acid amplification reaction in one or more positive controls, and to generate a determination result for the biological sample on the basis of the detection result of the nucleic acid amplification reaction in the one or more positive controls and a detection result of the nucleic acid amplification reaction in the biological sample. For example, a delay of rising of a fluorescence signal due to a matrix effect can be detected by determining whether to adjust the nucleic acid amplification reaction, and reaction control such as prolongation of a nucleic acid amplification reaction time can be performed. Therefore, an appropriate calibration curve can be created, and moreover, an appropriate determination can be made on the basis of the created calibration curve.

The control unit may be configured to generate determination reference data on the basis of the detection result of the nucleic acid amplification reaction in the one or more positive controls, and then, determine whether to adjust the nucleic acid amplification reaction on the basis of the determination data. The determination reference data is data to be referred to for determining whether a target nucleic acid is present in the biological sample or identifying an abundance of the target nucleic acid. For example, the control unit can determine whether the target nucleic acid is present in the biological sample and/or identify the abundance of the target nucleic acid on the basis of the detection result of the nucleic acid amplification reaction in the biological sample and the determination reference data.

In the present specification, a “detection result of a nucleic acid amplification reaction” includes detection result data regarding a signal (particularly, the fluorescence signal) detected when the nucleic acid amplification reaction is performed. The detection result data may be, for example, data that can be output as a plot in which an amplification time (in the case of isothermal nucleic acid amplification) or the number of amplification cycles (in the case of PCR) is a horizontal axis and a signal value is a vertical axis.

In the present specification, a “positive control” refers to a sample known to contain a nucleic acid that needs to be amplified in a case where a nucleic acid amplification reaction is executed. The nucleic acid may be, for example, a primer used for isothermal nucleic acid amplification or a probe used in a PCR reaction. The amount of the nucleic acid contained in the sample may also be known. Note that there is also a case where the positive control refers to a well containing the sample in the present specification.

In the present specification, a “negative sample” may be another sample determined to be negative. That is, a “detection result of a nucleic acid amplification reaction in a negative sample” may be a detection result of a nucleic acid amplification reaction executed on another sample determined to be negative, and particularly, may be a detection result of a nucleic acid amplification reaction that is the basis for determining that the another sample is negative.

The “nucleic acid amplification reaction executed on the another sample determined to be negative” may be another nucleic acid amplification reaction other than the nucleic acid amplification reaction executed on the biological sample to be analyzed, and more specifically, may be another nucleic acid amplification reaction that has been already executed before the nucleic acid amplification reaction executed on the biological sample is started.

In the present disclosure, it is possible to determine whether a signal is a signal caused by occurrence of non-specific amplification or a signal caused by a fluctuation of a Ct value that may occur in a case of a low copy number by referring to the detection result of the nucleic acid amplification reaction in the negative sample.

A type of the another nucleic acid amplification reaction is preferably the same as a type of the nucleic acid amplification reaction executed on the biological sample. For example, in a case where the nucleic acid amplification reaction executed on the biological sample is an isothermal nucleic acid amplification reaction (for example, LAMP), the another nucleic acid amplification reaction is also the isothermal nucleic acid amplification reaction (for example, LAMP).

Furthermore, in order to unify reaction conditions and detection conditions, a type of a reagent used in the another nucleic acid amplification reaction may be preferably the same as a type of a reagent used in the nucleic acid amplification reaction executed on the biological sample.

Furthermore, in order to unify the reaction conditions and the detection conditions, a type of a nucleic acid amplification device used to execute the another nucleic acid amplification reaction may be preferably the same as a type of a nucleic acid amplification device used in the nucleic acid amplification reaction executed on the biological sample. For example, two nucleic acid amplification devices of the same type or similar types may be used in the nucleic acid amplification reaction executed on the biological sample and the another nucleic acid amplification reaction, respectively.

Furthermore, in order to unify the reaction conditions and the detection conditions, a type of a container used to store the another sample in the another nucleic acid amplification reaction may be preferably the same as a type of a container used to store the biological sample in the nucleic acid amplification reaction executed on the biological sample. For example, chips of the same type and containing the same reagent may be used in these two reactions.

The another sample and the another nucleic acid amplification reaction may be appropriately selected by those skilled in the art according to the biological sample and the nucleic acid amplification reaction executed on the biological sample.

Note that a reaction time (in the case of isothermal nucleic acid amplification) or the number of reaction cycles (in the case of PCR) of the another nucleic acid amplification reaction may be different from those of the nucleic acid amplification reaction executed on the biological sample. This is because there is a possibility that the reaction time is regulated in analysis using the nucleic acid analysis system of the present disclosure.

The determination reference data preferably includes calibration curve data. The calibration curve data may include a mathematical formula representing the calibration curve, or may include parameters (for example one or more coefficients, particularly a slope, an intercept, and the like) constituting the mathematical formula.

The control unit may determine whether to adjust the nucleic acid amplification reaction, for example, on the basis of the determination reference data and standard reference data. The standard reference data is standard determination reference data (particularly, standard calibration curve data), and may be prepared in advance. The standard reference data may include, for example, ideal calibration curve data or average calibration curve data generated on the basis of a plurality of pieces of determination reference data.

The control unit may compare the determination reference data with the standard reference data to identify whether a delay of the nucleic acid amplification reaction (particularly the delay of rising of the fluorescence signal) due to the matrix effect has occurred and a degree of the delay, and then, determine whether to adjust the nucleic acid amplification reaction.

In one embodiment of the present disclosure, the control unit executes a determination result generation process of generating a determination result for the biological sample, and executes a wrong answer determination process of determining whether a determination result regarding whether a target nucleic acid is detected in the biological sample is a wrong answer in the determination result generation process. The wrong answer determination process may be, for example, a process of determining whether it is a false positive in a case where a determination result that is positive is obtained, or a process of determining whether it is a false negative in a case where a determination result that is negative is obtained.

In a particularly preferred embodiment, the control unit may be configured to execute the wrong answer determination process in a case where the detection result of the nucleic acid amplification reaction in the biological sample does not satisfy a predetermined condition. The predetermined condition may be set according to, for example, the likelihood of occurrence of a variation in the Ct value or a Tt value. The predetermined condition may be, for example, a condition that “the amount of the target nucleic acid contained in the biological sample is a predetermined value or more”. In a case where this condition is not satisfied, that is, in a case where the amount of the target nucleic acid contained in the biological sample is less than the predetermined value, the control unit may execute the wrong answer determination process. As described above, the variation in the Ct value or the Tt value is likely to occur in a case where the amount of the target nucleic acid is small, and thus, processing can be made efficient by executing the wrong answer determination process only in such a case.

### (2) Example of Nucleic Acid Analysis System

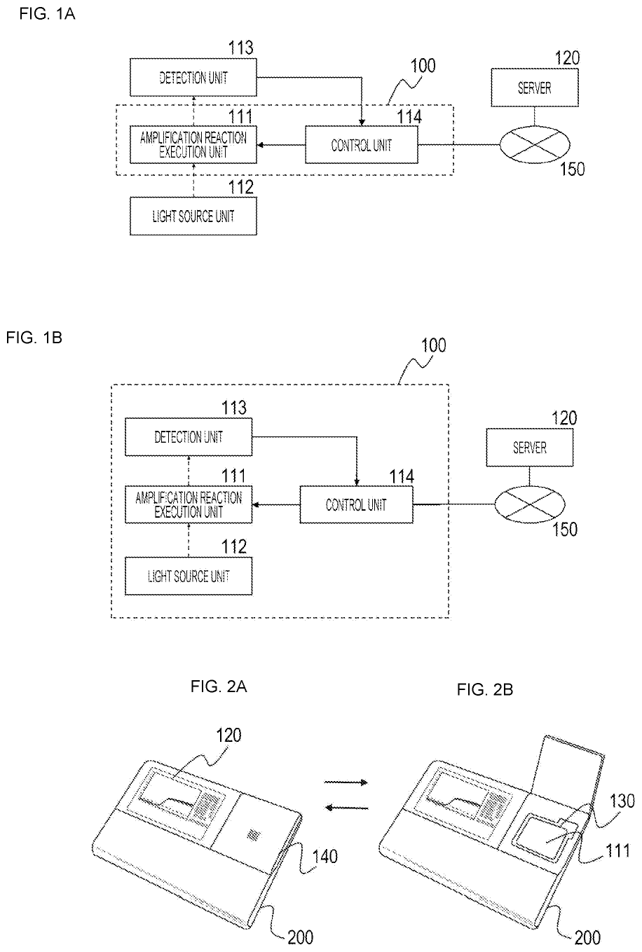

FIG. 1A illustrates a configuration example of the nucleic acid analysis system according to the present disclosure. A nucleic acid analysis system 100 illustrated in the drawing includes an amplification reaction execution unit 111 and a control unit 114. Furthermore, in a case where nucleic acid analysis is performed by the nucleic acid analysis system, a light source unit 112 that irradiates a sample group to be analyzed with light and a detection unit 113 that detects light generated in a nucleic acid amplification reaction in the sample group may be used. In the present disclosure, the amplification reaction execution unit 111 and the control unit 114 may be provided in one nucleic acid amplification device. Alternatively, the amplification reaction execution unit 111 may be provided in the nucleic acid amplification device, and the control unit 114 may be provided in an information processing device.

Note that the nucleic acid analysis system 100 may be configured to include the amplification reaction execution unit 111, the light source unit 112, the detection unit 113, and the control unit 114 as illustrated in FIG. 1B.

Each of these components will be described hereinafter.

### (2-1) Amplification Reaction Execution Unit

The amplification reaction execution unit 111 is configured to execute a nucleic acid amplification reaction for a biological sample. The amplification reaction execution unit 111 may execute temperature regulation that enables amplification of a target nucleic acid that may be contained in the biological sample. The nucleic acid amplification reaction may be an isothermal nucleic acid amplification reaction or a polymerase chain reaction (PCR).

The isothermal nucleic acid amplification reaction may be any one of loop-mediated isothermal amplification (LAMP), whole genome amplification (WGA), multiple displacement amplification (MDA), strand displacement amplification (SDA), nicking endonuclease amplification reaction (NEAR), helicase-dependent amplification (HDA), recombinase polymerase amplification (RPA), strand-invasion based amplification (SIBA), nucleic acid sequences based amplification (NASBA), and transcription mediated amplification (TMA), and is particularly loop-mediated isothermal amplification (LAMP).

The PCR may be particularly real-time PCR (quantitative PCR).

For example, in a case where the nucleic acid amplification reaction is isothermal nucleic acid amplification reaction, the amplification reaction execution unit 111 executes temperature regulation for maintaining the temperature of the biological sample at a predetermined temperature. The predetermined temperature may be appropriately set by those skilled in the art according to a type of the isothermal amplification reaction.

Furthermore, in a case where the nucleic acid amplification reaction is the polymerase chain reaction (PCR), the amplification reaction execution unit 111 regulates the temperature of the biological sample such that a predetermined cycle (a denaturation step, an annealing step, and an elongation step) is executed a plurality of times. The temperature in each step included in the cycle may be appropriately selected by those skilled in the art.

For example, the amplification reaction execution unit 111 may be configured to be capable of accommodating a container including the biological sample, and may be configured to be capable of regulating the temperature of the biological sample in the container.

For example, the nucleic acid analysis system 100 may include a nucleic acid amplification device 200, for example, as illustrated in A and B of FIG. 2. The nucleic acid amplification device 200 includes the amplification reaction execution unit 111, the light source unit 112, the detection unit 113, and the control unit 114. That is, in one embodiment of the present disclosure, the nucleic acid analysis system may include a nucleic acid amplification device including the amplification reaction execution unit 111 and the control unit 114. The nucleic acid amplification device may further include the light source unit 112 and/or the detection unit 113.

A of FIG. 2 illustrates a state in which a lid 140 of a container accommodating portion 130 of the device is closed, and B of FIG. 2 illustrates a state in which the lid 140 is opened. The container is accommodated in the container accommodating portion 130, and then, the lid 140 is closed. After the lid 140 is closed, the amplification reaction execution unit 111 starts a temperature regulation process of the sample group (including, for example, the biological sample and one or more positive controls) in the container, for example, in response to a user pressing a button configured to start a nucleic acid amplification process or selecting an icon for the start. By the temperature regulation, the nucleic acid amplification reaction occurs.

More specifically, the amplification reaction execution unit 111 includes a temperature regulation device that performs the temperature regulation of the biological sample in the container. This device may be, for example, a transparent conductive film such as a light-transmissive ITO heater. As the temperature regulation device, a device known in the art may be adopted.

### (2-2) Light Source Unit

The light source unit 112 is configured to irradiate the biological sample with light. By the light irradiation, a fluorescent substance for detecting nucleic acid amplification in the biological sample is excited to generate fluorescence. In particular, an amplification product in the biological sample, in particular, the fluorescent substance contained in the amplification product is excited by the light irradiation to generate the fluorescence. The fluorescence is detected by the detection unit 113.

The light source unit 112 includes one or more light sources that perform the light irradiation. The one or more light sources include an excitation light emitting light source that emits excitation light for the fluorescent substance. The excitation light emitting light source may be, for example, one or more of a laser light source, an LED light source, a mercury lamp, and a tungsten lamp. One of these may be used alone, or a plurality of these may be used in combination. In particular, one or a plurality of laser light sources and/or LED light sources is suitable as the excitation light emitting light source.

The light source unit 112 may include a turbidity measurement light source that emits turbidity measurement light in order to perform melting curve analysis to be described later. The turbidity measurement light source is preferably a light source that emits light having a wavelength matching a fluorescence wavelength of the fluorescent substance contained in the amplification product or light in a wavelength range including the fluorescence wavelength. The turbidity measurement light source may be, for example, light that has various wavelengths being mixed and may be emitted from a light source such as a deuterium lamp, a tungsten lamp, a xenon lamp, a mercury lamp, or a halogen lamp, or may be a laser light source and/or an LED light source. The turbidity measurement light enables, for example, analysis of nucleic acid amplification that utilizes turbidity described in Patent Document 3.

The light source unit 112 may further include a light guide optical system configured to guide the light emitted from the excitation light emitting light source to the biological sample. The configuration of the light guide optical system may be appropriately designed by those skilled in the art. The light guide optical system may include, for example, a light guide member (for example, a light guide plate) into which light emitted from the light source enters, and various optical elements (for example, a condenser lens and the like) causing the light emitted from the light guide member to travel to and be collected on the biological sample.

### (2-3) Detection Unit

The detection unit 113 is configured to detect the fluorescence generated from the fluorescent substance for detecting the nucleic acid amplification. The detection unit 113 may include a light detection device, and the light detection device may be, for example, an area imaging element such as a CCD or a CMOS, a photomultiplier tube (PMT), a photodiode, or the like.

Light detection for performing the melting curve analysis to be described later may also be performed by the detection unit 113 configured for the fluorescence detection. The detection unit 113 may detect scattered light or transmitted light generated by irradiation of the biological sample with light by the light source unit 112.

The detection unit 113 may further include an optical element for causing light that needs to be detected to reach the light detection device. The optical element may be disposed between the biological sample and the light detection device. The optical element may be, for example, an optical filter (particularly, a fluorescence filter) and/or a lens (particularly, a condenser lens).

### (2-4) Control Unit

The control unit 114 controls driving of the amplification reaction execution unit 111, the light source unit 112, and the detection unit 113. The control unit 114 may be configured as a general-purpose computer including, for example, a CPU, a memory, a hard disk, and the like.

The control unit 114 may be configured to be connectable to a network 150 in a wired or wireless manner. The control unit 114 may be configured to be capable of transmitting various types of data to a server 120 via the network 150 or receiving various types of data from the server 120 via the network 150.

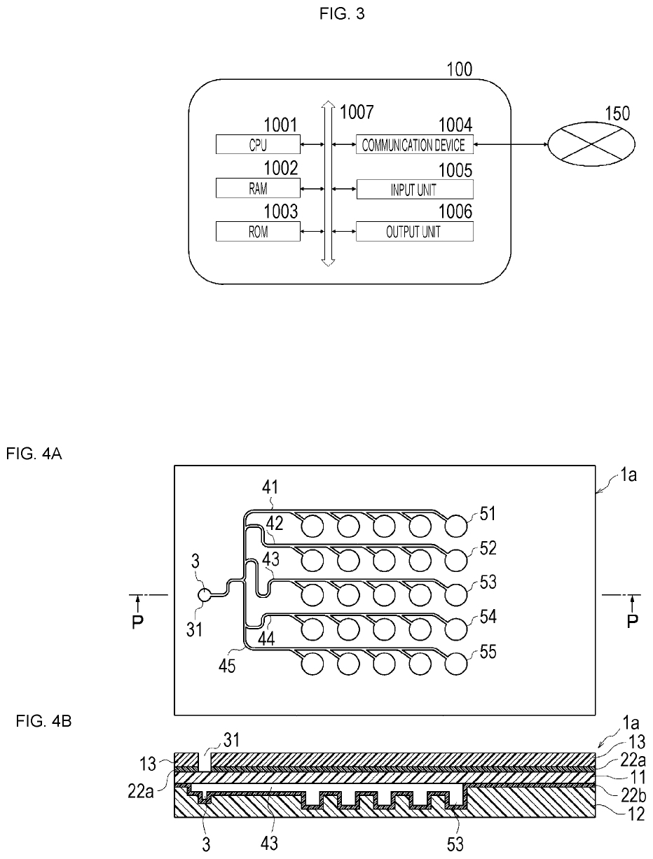

A specific configuration example of the control unit 114 will be described hereinafter with reference to FIG. 3.

As illustrated in the drawing, the control unit 114 includes a central processing unit (CPU) 1001 and a RAM 1002. The CPU 1001 and the RAM 1002 are connected to each other via a bus 1007, and are also connected to other components of the control unit 114 via the bus 1007.

The CPU 1001 performs control and/or arithmetic processing executed by the control unit 114. An arbitrary processor can be used as the CPU 1001, and examples thereof include processors of Xeon (registered trademark) series, Core (trademark) series, or Atom (trademark) series. A function executed by the control unit 114 may be implemented by, for example, the CPU 1001.

The RAM 1002 may include, for example, a cache memory and a main memory, and temporarily store a program used by the CPU 1001.

The control unit 114 may include a ROM 1003, a communication device 1004, and a drive (not illustrated). Any of these components may be connected to the bus 1007.

The ROM 1003 may store an operating system (for example, WINDOWS (registered trademark), UNIX (registered trademark), LINUX (registered trademark), or the like), a program for causing a device (such as the nucleic acid amplification device or the information processing device) to execute processing according to the present disclosure, and other various programs, and various types of data (for example, data mentioned in the present specification such as the detection result data and the determination reference data). The ROM 1003 may be, for example, a semiconductor recording medium such as a flash memory, but is not limited thereto.

The communication device 1004 connects the control unit 114 to the network 150 in a wired or wireless manner. The control unit 114 can acquire various types of data (for example, video data) via the network 150 using the communication device 1004. The acquired data may be stored in, for example, the ROM 1003. A type of the communication device 1004 may be selected as appropriate by those skilled in the art.

The drive can read information recorded on a recording medium and output the information to the RAM 1003. The recording medium is, for example, a microSD memory card, an SD memory card, or a flash memory, but is not limited to these.

The control unit 114 also includes an input unit 1005 and an output unit 1006. The input unit 1005 may be configured to receive data input from the outside of the control unit 114, and the configuration thereof may be appropriately selected by those skilled in the art. The output unit 1006 may be configured to be capable of outputting data included in the control unit 114, and may be configured to be capable of outputting data to, for example, a display device or a printing device. The output unit 1006 itself may include the display device. For example, as illustrated in FIG. 2, the nucleic acid amplification device 200 included in the nucleic acid analysis system according to the present disclosure may have a display device 120 as the output unit 1006. The display device 120 displays a detection result in the nucleic acid amplification reaction. Furthermore, the display device 120 may output a determination result generated by information processing according to the present disclosure. Therefore, the user can easily know the determination result by confirming a determination result image displayed by the display device 120.

### (2-5) Container

The biological sample may be subjected to the nucleic acid amplification reaction by the amplification reaction execution unit 111 in a state of being accommodated in the container such as a chip or a well plate. The container includes one or more reaction regions in which the nucleic acid amplification reaction for the one or more positive controls is executed and one or more reaction regions in which the nucleic acid amplification reaction for the biological sample is executed. The container may further include one or more reaction regions in which a nucleic acid amplification reaction for one or more negative controls is executed.

Examples of the container include a microchip described in Patent Document 1 and a microchip described in Patent Document 2. These microchips include a plurality of wells, and temperature regulation for causing a nucleic acid amplification reaction is executed on samples contained in these wells.

Hereinafter, a chip which is an example of the container will be described with reference to FIG. 4, and assignment of each of the reaction regions in the chip will be further described. The drawing is a schematic view illustrating a configuration of a chip 1. A of the drawing is a schematic top view, and B of the drawing is a schematic cross-sectional view corresponding to a cross section taken along line P-P in A of the drawing.

The chip 1 (also referred to as a microchip) illustrated in the drawing is provided with: an entry portion 3 which is a region where the biological sample that is liquid is introduced from the outside of the chip; wells 51 to 55 which are reaction sites of an analyte; and flow channels 41 to 45 connecting the entry portion 3 and the wells 51 to 55, respectively. In FIG. 2 and the description thereof, five wells to which the liquid is supplied through the flow channel 41 will be described as the wells 51, and similarly, five wells to which the liquid is supplied through each of the flow channels 42, 43, 44, and 45 will be described as each of the wells 52, 53, 54, and 55. Note that the configuration of the microchip is not limited to numbers and arrangements of the entry portion 3, the flow channels 41 to 45, and the wells 51 to 55 illustrated in the drawing.

The microchip includes a plurality of substrate layers and one or more bonding layers including a silicon compound and provided at boundary surfaces between the substrate layers. Furthermore, at least one of the bonding layers includes an organic silicon compound. The microchip 1 illustrated in B of the drawing includes, for example, three substrate layers 11, 12, and 13. The substrate layers 11, 12, and 13 preferably include substrate layers including non-silicone resins and a substrate layer including polydimethylsiloxane. For example, both surfaces of the substrate layer including polydimethylsiloxane may be bonded to a substrate layer including a first non-silicone resin and a substrate layer including a second non-silicone resin via the bonding layers, respectively. In the microchip 1, for convenience of description, the substrate layer including polydimethylsiloxane will be referred to as the substrate layer 11, and out of the two substrate layers bonded to the substrate layer 11, the “substrate layer including the first non-silicone resin” will be referred to as the “substrate layer 12”, and the “substrate layer including the second non-silicone resin” will be referred to as the “substrate layer 13”.

In the microchip 1, the substrate layer 12 has a trench on a bonding surface with the substrate layer 11, and the trench corresponds to a region of the entry portion 3, the flow channels 41 to 45, and the wells 51 to 55 into which the biological sample is introduced. The substrate layer 12 is bonded to the substrate layer 11 via a bonding layer 22b including a silicon compound. The bonding layer 22b may be a bonding layer including an inorganic silicon compound. On the other hand, the substrate layer 13 has no trench on a bonding surface with the substrate layer 11, and the substrate layer 13 may be bonded to the substrate layer 11 via a bonding layer 22a including an organic silicon compound.

In the microchip 1, the trench formed in the substrate layer 12 is provided on the bonding surface with the substrate layer 11. Therefore, the region into which the biological sample is introduced, such as the entry portion 3, is not in communication with the outside of the microchip 1. In a case where the substrate layer 11 includes an elastic material, a part of a puncture member, such as a needle, can penetrate the entry portion 3 from the outside of the microchip 1 through an inlet 31 formed in the substrate layer 13. When a syringe or the like to which the needle is connected is filled with the biological sample in advance and the needle penetrates the substrate layer 11, the biological sample can be introduced into the region such as the entry portion 3 in the microchip 1. Moreover, since the region that has been sealed is connected only to the inside of the syringe by the penetration of the puncture member, the biological sample can be introduced without causing air bubbles to enter the flow channels 41 to 45 or the wells 51 to 55.

The inside of the chip 1 (that is, the wells and the flow channels) may have a negative pressure relative to atmospheric pressure. Therefore, the biological sample passes through the inside of the flow channel and reaches each of the wells when the entry portion 3 is pierced with the needle. The biological sample may move automatically by the negative pressure.

When the puncture member is removed from the entry portion 3 after the liquid is introduced, a puncture point can be naturally sealed by a self-sealing property of the substrate layer 11 if the substrate layer 11 includes the elastic material. The natural sealing of the puncture point of the needle or the like due to elastic deformation of the substrate layer is also referred to as the “self-sealing property” of the substrate layer. In this manner, the substrate layer 11 preferably includes a material having the self-sealing property.

The biological sample introduced into the microchip 1 may be a biological sample that is likely to contain a target nucleic acid to be analyzed. The target nucleic acid may be DNA or RNA. The biological sample may be a nasal or pharyngeal swab, a liquid biological sample such as the swab, saliva, blood, or body fluid, or a solution containing these liquid biological samples (for example, a diluent or the like).

A part of the wells 51 to 55 included in the microchip 1 may be assigned as wells in which the nucleic acid amplification reaction for the one or more positive controls is performed, another part of the wells may be assigned as wells for detecting the target nucleic acid in the biological sample, and all the remaining wells may be assigned as wells in which the nucleic acid amplification reaction does not occur in a case where the target nucleic acid is present in the biological sample.

Alternatively, a part of the wells 51 to 55 included in the microchip 1 may be assigned as wells in which the nucleic acid amplification reaction for the one or more positive controls is performed, another part of the wells may be assigned as wells for detecting the target nucleic acid in the biological sample, and all the remaining wells may be assigned as wells for detecting another target nucleic acid in the biological sample.

Alternatively, some of the wells 51 to 55 included in the microchip 1 may be assigned as wells in which the nucleic acid amplification reaction for the one or more positive controls is performed, and all the other wells may be assigned as wells for detecting the target nucleic acid in the biological sample.

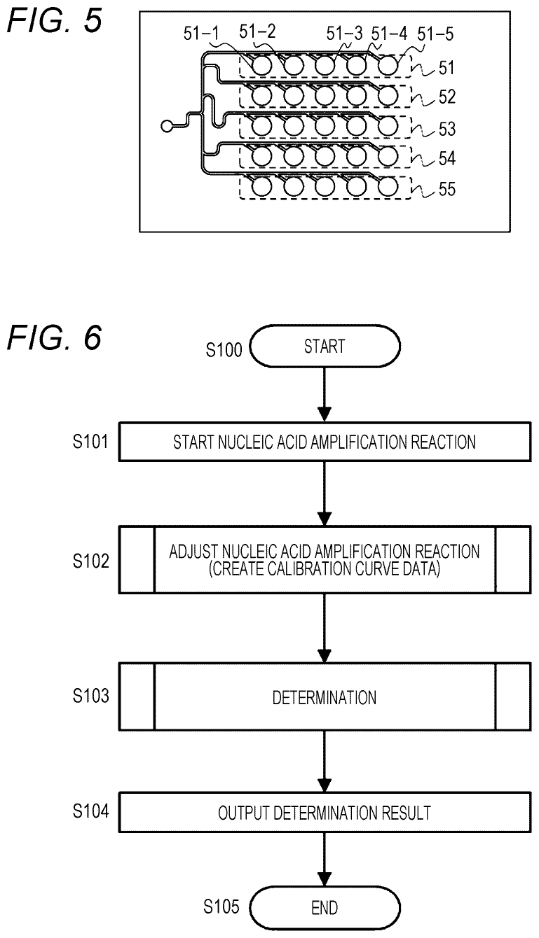

For example, in a case where the nucleic acid amplification reaction is isothermal nucleic acid amplification such as an LAMP method, wells used as positive controls may contain mutually different copy numbers of primers. For example, the five wells 51 illustrated in FIG. 5 are assigned as wells in which the nucleic acid amplification reaction for the one or more positive controls is executed, and the other wells 52 to 55 are assigned as wells in which the nucleic acid amplification reaction of the target nucleic acid is executed, wells in which an amplification reaction of another target nucleic acid is executed, or wells (also referred to as negative control wells) in which no amplification reaction occurs in a case where the target nucleic acid is amplified.

In the present disclosure, an initial template amount in the nucleic acid amplification reaction may be adjusted by adjusting the amounts (copy numbers) of the primers contained in the wells assigned as the positive controls. Therefore, the wells assigned as the positive controls may contain, for example, the mutually different copy numbers of primers. The copy number of the primer contained in each well can be set to increase, for example, at a predetermined common ratio. For example, regarding the five wells 51 illustrated in FIG. 5, wells 51-5, 51-4, 51-3, 51-2, and 51-1 may contain primers of copy numbers of 10⁵, 10⁴, 10³, and 10², and 10¹, respectively. For example, in the case of the LAMP method, the primers contained in the wells forming the positive controls in mutually different copy numbers may be one or more primers selected from among four types of primers of a forward inner primer (FIP), a F3 primer, a backward inner primer (BIP), and a B3 primer, and particularly, is the F3 primer.

In the present disclosure, a group of wells having initial templates at mutually different concentrations may be prepared to create a calibration curve.

Note that a plurality of types of primers for amplifying a predetermined nucleic acid is also referred to as a primer set in the present specification.

The primer set contained in the positive control well may be a primer set for amplifying a nucleic acid whose presence in the biological sample is known or certain. As the nucleic acid, for example, a nucleic acid for amplification of an endogenous normalizer gene generally used in the art may be adopted. Examples of the endogenous normalizer gene can include a β-actin gene (ACTB), a 18S ribosomal RNA gene (rRNA), a cyclophilin A gene (CYC), a glyceraldehyde phosphate dehydrogenase gene (GAPDH), a β-2 microglobulin gene (B2M), a β-glucuronidase gene (GUS), a hypoxanthine ribosyltransferase gene (HPRT), a TATA-Box binding protein gene (TBP, and the like. The nucleic acid is a nucleic acid other than the target nucleic acid to be analyzed.

The primer set contained in the wells in which the nucleic acid amplification reaction of the target nucleic acid is executed may be a primer set for amplifying the target nucleic acid. The target nucleic acid is, for example, a nucleic acid contained in a virus (for example, a coronavirus or an influenza virus) or a bacterium, and is particularly a nucleic acid specific to a virus or a bacterium that needs to be detected. The target nucleic acid may be appropriately selected by those skilled in the art.

A primer sequence in the well forming the positive control can be appropriately set by those skilled in the art according to a nucleic acid sequence to be amplified in the same well or the like, and software known in the art may be used for designing the primer sequence.

Similarly, those skilled in the art can appropriately set a primer sequence contained in the well in which the nucleic acid amplification reaction of the target nucleic acid is executed.

### (2-5-1) Examples of Primers Used in LAMP Method

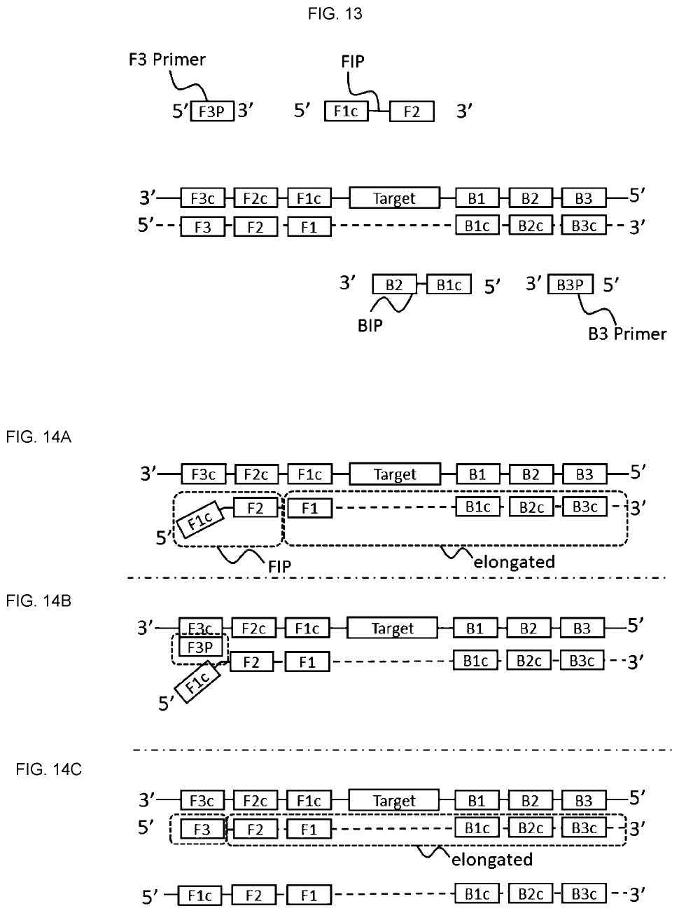

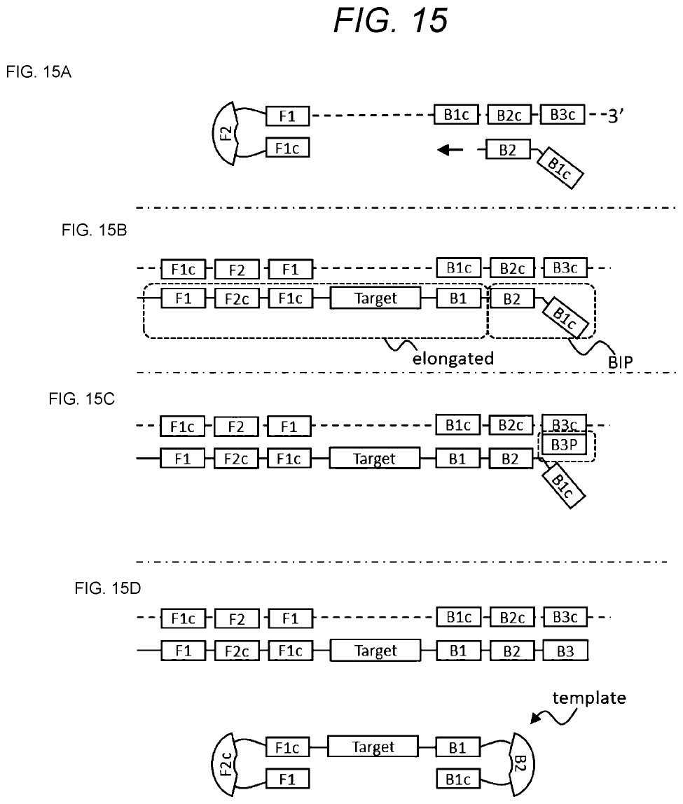

The primers used in the LAMP method will be described with reference to FIG. 13 to 15. FIG. 13 is a schematic view for describing the primers. FIGS. 14 and 15 are views for describing that an initial template is formed using the primers.

As illustrated in FIG. 13, in the LAMP method, six primer binding sites are set around the target nucleic acid. As illustrated in FIG. 13, binding sites F3c, F2c, and F1c and B1, B2, and B3 are set around the target nucleic acid (Target) from the 3′ terminal side. Furthermore, binding sites F3, F2, and F1, and B1c, B2c, and B3c are set around a complementary strand of the target nucleic acid. The FIP includes F2 and F1c. The F3 primer has the same sequence as F3. The BIP includes B2 and B1c. The B3 primer has the same sequence as B3.

When the isothermal nucleic acid amplification is started, the target nucleic acid and the complementary strand are dissociated from each other to form a single strand. Then, as illustrated in a of FIG. 14, the FIP complementarily binds to F2c on the 3′ side of the target nucleic acid, and an elongation reaction occurs. Next, as illustrated in b of the drawing, the F3 primer complementarily binds to F3c on the 3′ side of the target nucleic acid, and an elongation reaction occurs. Therefore, as illustrated in c of the drawing, the chain elongated starting from the FIP is dissociated from the target nucleic acid. The chain elongated starting from the FIP may form a loop by F1 and F1c as illustrated in d of FIG. 15.

Next, as illustrated in d and e of FIG. 15, the BIP complementarily binds to B2c of the strand elongated starting from the FIP, and an elongation reaction occurs. Next, as illustrated in f of the drawing, the B3 primer complementarily binds to B3c, and an elongation reaction occurs. Therefore, as illustrated in g of the drawing, the chain elongated starting from the BIP is dissociated to be a single strand. At both ends of the single strand, there are a complementary combination of F1 and F1c and a complementary combination of B1 and B1c. Therefore, these complementary combinations may be bound to form a dumbbell structure as illustrated in g of the drawing. The single strand is used as a template to be amplified in the LAMP method. Since the template to be amplified is formed in this manner, for example, the amount (copy number) of the initial template present at the beginning of a reaction in a nucleic acid amplification reaction in a positive control can be controlled by the number of the F3 primers.

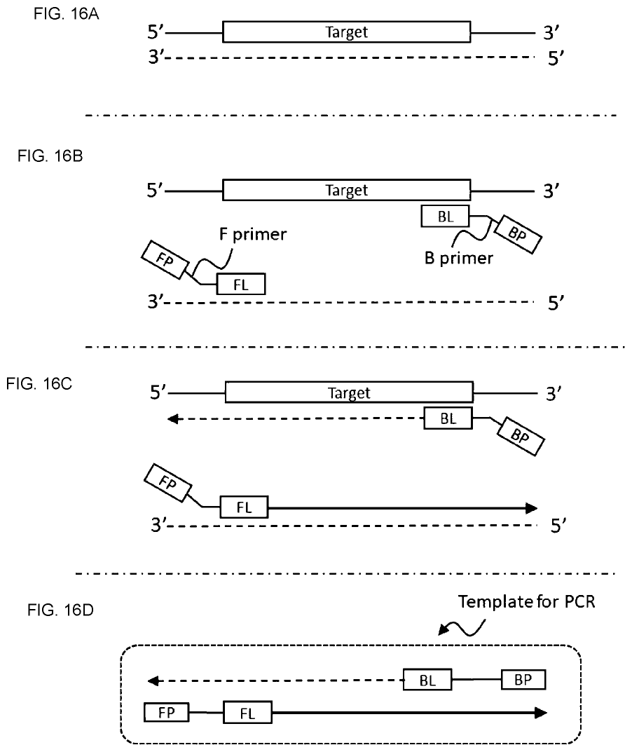

Furthermore, a primer used in a case where PCR is adopted as a nucleic acid amplification reaction and the nucleic acid amplification reaction using the primer in the present disclosure will be described below with reference to FIG. 16. In this case, each well may contain two types of a first DNA polymerase to perform isothermal nucleic acid amplification and a second DNA polymerase to perform PCR in each well. Note that the first DNA polymerase may be an enzyme that is deactivated at a temperature (for example, about 95° C.) at which double-stranded DNA is converted into single-stranded DNA by thermal denaturation.

### (2-5-2) Examples of Primers Used in PCR

In an example of a nucleic acid amplification reaction in the case of adopting PCR, it is assumed that a target nucleic acid (Target) as illustrated in a of FIG. 16 is to be analyzed. In this case, as illustrated in b of the drawing, two types of primers are used: a B primer containing a primer sequence BL that complementarily binds to the 3′ terminal of the target nucleic acid and a primer sequence BP used in the PCR to be described later; and an F primer containing a primer sequence FL that complementarily binds to the 3′ terminal of a complementary strand of the target nucleic acid and a primer sequence FP used in the PCR to be described later.

A double strand of the target nucleic acid and its complementary strand illustrated in b of the drawing is elongated by the first DNA polymerase. In the elongation, the primer sequence BL contained in the B primer complementarily binds to the target nucleic acid, and then, a nucleic acid chain is elongated. Furthermore, the primer sequence FL contained in the F primer complementarily binds to the complementary strand of the target nucleic acid, and then, a nucleic acid chain is elongated. Here, an elongation reaction by the first DNA polymerase can be performed, for example, at 63° C. These elongation reactions occur to form a state as illustrated in c of the drawing. In this state, the FP contained in the F primer is not complementary to a sequence of a strand containing the target nucleic acid, and thus, does not bind to the strand. Furthermore, the BP contained in the B primer is not complementary to a sequence of a strand containing the complementary strand, and thus, does not bind to the strand.

After the state as illustrated in c of the drawing is formed, thermal denaturation is performed. The thermal denaturation is performed at a temperature at which the first DNA polymerase is deactivated. For example, the temperature may be 80° C. to 100° C., particularly about 95° C. Such a temperature inactivates a DNA polymerase used in an isothermal nucleic acid amplification reaction, but does not inactivate a DNA polymerase used in a PCR reaction. That is, in the thermal denaturation, the second DNA polymerase is not deactivated.

By the thermal denaturation, the chain elongated starting from FL and the chain elongated starting from BL are dissociated from the template. Then, after the thermal denaturation, these two chains form a double strand as illustrated in d of the drawing. The double strand may be used as a template in the PCR reaction. Note that the PCR reaction may be performed without forming the double strand. The target nucleic acid is amplified by the PCR reaction. Then, the amplification is detected by, for example, an amplification detection technique known in the field of quantitative PCR.

As described above, the primer used in the case of adopting the PCR may contain both a primer sequence for amplification by an isothermal nucleic acid amplification enzyme (the first DNA polymerase, particularly an enzyme for the LAMP) and a primer sequence for amplification by a PCR enzyme (the second DNA polymerase).

### (3) Examples of Processes Executed by Nucleic Acid Analysis System

An example of a nucleic acid analysis process executed by the nucleic acid analysis system 100 will be described below with reference to FIG. 6. FIG. 6 is a flowchart of the process. Furthermore, this example is an example in which the chip 1 described with reference to FIG. 5 in the above (2) is used as a container in which a biological sample is stored. In the chip 1, the wells 51 are assigned as positive controls. Furthermore, the wells 52 are assigned as wells in which a signal increase is detected in a case where a target nucleic acid is present in the biological sample. The wells 53 to 55 are assigned as wells in which no signal increase is detected in a case where the target nucleic acid is present in the biological sample. Each well may contain a reagent group necessary for causing a nucleic acid amplification reaction in advance. The reagent group can be appropriately selected by those skilled in the art according to the target nucleic acid to be amplified and a type of the nucleic acid amplification reaction.

In step S100, the nucleic acid analysis system 100 starts the nucleic acid analysis process. The control unit 114 may start the process, for example, in response to a user pressing a start button of the process.

In step S101, the nucleic acid analysis system 100 starts a nucleic acid amplification reaction. In the same step, the control unit 114 controls the amplification reaction execution unit 111 to execute temperature regulation of the biological sample such that a predetermined nucleic acid amplification reaction occurs. As described above, the nucleic acid amplification reaction may be an isothermal nucleic acid amplification reaction or PCR.

In step S102, the nucleic acid analysis system 100 executes an adjustment process of the nucleic acid amplification reaction. The adjustment process may be executed while the amplification reaction execution unit 111 is executing the nucleic acid amplification reaction. In the adjustment process, the control unit 114 executes a process of generating determination reference data used in step S103. The control unit 114 may adjust the nucleic acid amplification reaction according to the generated determination reference data, and may adjust, for example, a reaction time or the number of reaction cycles of the nucleic acid amplification reaction. The adjustment of the reaction time is, for example, prolongation or shortening of the reaction time, particularly prolongation. The adjustment of the number of reaction cycles is, for example, an increase or decrease in the number of reaction cycles, particularly an increase.

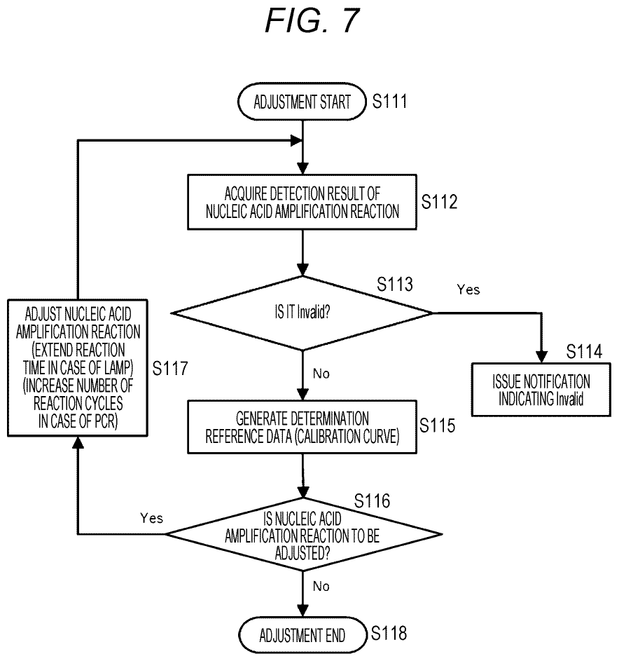

Details of the adjustment process will be described below (3-1) with reference to a flowchart of FIG. 7.

In step S103, the nucleic acid analysis system 100 (particularly, the control unit 114) executes a determination process. The determination process may include a process of determining whether the target nucleic acid is contained in the biological sample. The determination process may include a process of identifying the amount of the target nucleic acid contained in the biological sample.

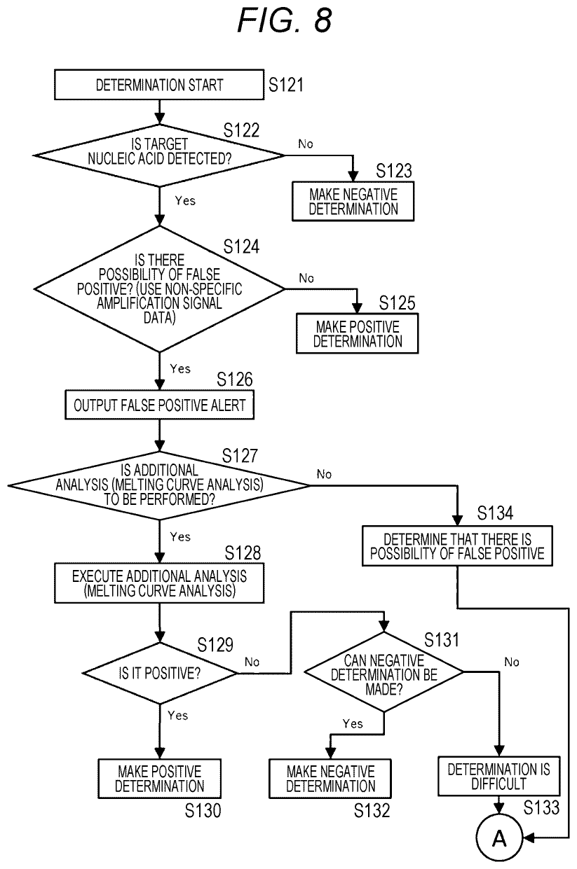

Details of the determination process will be described below in (3-2) and (3-3) with reference to flowcharts of FIG. 8 to 11.

In step S104, the nucleic acid analysis system 100 outputs a determination result generated in step S103. For example, a nucleic acid amplification device or an information processing device included in the nucleic acid analysis system 100 may include an output device (for example, a display device or a printing device), and the output device may output the determination result.

In step S105, the nucleic acid analysis system 100 ends the nucleic acid analysis process.

### (3-1) Nucleic Acid Amplification Reaction Adjustment Process

FIG. 7 illustrates an example of a flowchart of a process executed by the nucleic acid analysis system 100 (particularly, the control unit 114) in step S102 described above.

In step S111, the control unit 114 starts the nucleic acid amplification reaction adjustment process.

The control unit 114 starts the adjustment process, for example, after a predetermined time has elapsed since the start of the nucleic acid amplification reaction in step S101 (in the case of isothermal nucleic acid amplification) or after a predetermined number of cycles have been executed (in the case of PCR).

In step S112, the control unit 114 acquires a detection result of the nucleic acid amplification reaction (also referred to as “detection result data of the nucleic acid amplification reaction” in the present specification). The detection result data may be transmitted from the detection unit 113. The detection result data is data related to light generated by the detection unit 113 from each of the plurality of wells, and is particularly light signal data. The plurality of wells includes one or more positive control wells and one or more wells in which the nucleic acid amplification reaction of the target nucleic acid that may be contained in the biological sample is executed. The detection result data may be a result obtained by detecting the light at predetermined time intervals or at a plurality of predetermined time points. The light is light generated due to the occurrence of the nucleic acid amplification reaction, and is, for example, fluorescence.

For example, the control unit 114 acquires pieces of the detection result data of the nucleic acid amplification reactions in the wells 51 (positive control wells) and the wells 52 (biological sample wells). These pieces of detection result data are used in the following steps.

Furthermore, the control unit 114 may acquire pieces of detection result data of nucleic acid amplification reactions in the wells 53 to 55 (wells in which no signal increase is detected in a case where the target nucleic acid is present in the biological sample). The control unit 114 can transmit the detection result data to the server 120 via the network 150. The detection result data can be accumulated in the server 120. This detection result data may be used as a reference negative detection result to be described later.

In step S113, the control unit 114 determines whether the detection result is invalid. The control unit 114 makes the determination on the basis of the detection result of the nucleic acid amplification reaction regarding the positive control well. For example, the control unit 114 refers to the detection result of the nucleic acid amplification reaction regarding at least one positive control well, and determines that the detection result is invalid in a case where the detection result does not satisfy a predetermined condition.

For the determination, for example, only the detection result of the positive control well having the largest initial template copy number may be referred to, or the detection result of the positive control well having the second largest copy number or the detection results of the positive control wells having the second and third largest copy numbers may be referred to in addition to the positive control well having the largest copy number. For example, the control unit 114 may refer to only a detection result of the well 51-5 (copy number: 105), or may refer to the detection result of the well 51-4 (copy number: 104) or/and the detection result of the well 51-3 (copy number: 103) in addition to the detection result of the well 51-5.

The predetermined condition may be whether the detection result data has reached a predetermined threshold, and more specifically, may be whether a fluorescence signal in a positive control at a predetermined time point after the start of the nucleic acid amplification reaction (for example, a value of the fluorescence signal at the predetermined time point or an average value of the fluorescence signals over a predetermined time) has reached the predetermined threshold.

The control unit 114 may determine that the detection result is invalid in a case where a detection result of a fluorescence signal in a positive control well is less than the predetermined threshold (or equal to or less than the threshold) at the time point when the predetermined time has elapsed since the start of the nucleic acid amplification reaction. On the other hand, the control unit 114 may determine that the detection result is not invalid (or valid) in a case where the detection result is equal to or more than the predetermined threshold (or more than the threshold). In a case where a plurality of positive control wells is referred to, the detection result may be determined to be invalid in a case where any one, two, or all of the positive controls do not satisfy the predetermined condition.

As described above, in step S113, the control unit 114 acquires the detection result of the nucleic acid amplification reaction in the positive control at the predetermined time point after the start of the nucleic acid amplification reaction, and determines that the detection result is invalid in a case where an increase in a light signal cannot be confirmed in the detection result. When a nucleic acid amplification reaction is started in step S101, typically, the nucleic acid amplification reaction occurs in a positive control, and fluorescence is generated with the amplification. However, there is a case where no nucleic acid amplification reaction occurs in the positive control, for example, in a case where a large amount of impurities (for example, blood or the like) is mixed in some processing performed before the nucleic acid amplification reaction is executed or the like. In such a case, there is a high possibility that determination reference data to be used in the determination process to be described later is not appropriately generated so that the determination becomes impossible, or a determination result is not valid even if the determination is executed. Therefore, when an invalidity determination process in step S113 is executed, it is possible to determine that the nucleic acid amplification reaction itself started in step S101 is invalid, and to prevent unnecessary processing from being executed.

In step S113, in a case where the control unit 114 does not determine that the detection result is invalid, the process proceeds to step S114. In a case where the control unit 114 determines that the detection result is invalid, the process proceeds to step S115. In this manner, the control unit 114 determines that the nucleic acid amplification reaction for the biological sample is invalid in a case where the determination reference data cannot be generated.

In step S114, the control unit 114 outputs a notification that the determination result is invalid. For example, the control unit 114 causes the output device (for example, the display device or the printing device) connected to the control unit 114 to output the notification.

In step S114, the control unit 114 may stop the nucleic acid amplification process by the amplification reaction execution unit 111, and particularly, the temperature regulation process of the biological sample may be stopped.

Alternatively, in step S114, the control unit 114 may display a screen asking the user whether to stop the nucleic acid amplification process. Then, the nucleic acid amplification process may be stopped in response to reception of an instruction to stop the nucleic acid amplification process through the screen.

In step S115, the control unit 114 generates the determination reference data on the basis of the detection result of the nucleic acid amplification reaction in one or more positive controls. The determination reference data is data to be referred to for determining whether the target nucleic acid is present in the biological sample to be analyzed. Furthermore, the determination reference data may be referred to in order to identify the amount (copy number) of the target nucleic acid in the biological sample.

The one or more positive controls may be a group of positive controls having mutually different copy numbers, and particularly, may be prepared as a group of positive controls having copy numbers diluted stepwise. The group of positive controls may be a group of samples diluted at predetermined dilution ratios, and may be, for example, a group of samples diluted stepwise at any common ratio of 2 to 20 (for example, a common ratio of 10). In one embodiment, the group of positive controls includes five samples containing nucleic acids for positive controls (for example, primers) each having a copy number of 10⁵, 10⁴, 10³, 10², or 10¹.

The determination reference data preferably includes calibration curve data. The control unit 114 may generate the calibration curve data on the basis of a Tt value or a Ct value of the nucleic acid amplification reaction in the one or more positive controls. In particular, the control unit 114 generates the calibration curve data on the basis of the Tt value or the Ct value and the amount of a nucleic acid for generating a calibration curve (for example, specific primer) contained in each of the one or more positive controls. The calibration curve data may be any data that enables execution of the determination, and does not necessarily include graphical data called a calibration curve. The calibration curve data may be mathematical formula data representing the calibration curve, and may include data related to a coefficient (for example, a slope) included in the mathematical formula representing the calibration curve.

In one embodiment of the present disclosure, the control unit 114 may generate the determination reference data (particularly, calibration curve data) using a trained model. The trained model may be, for example, a trained model generated by machine learning in which a plurality of combinations of a detection result of a nucleic acid amplification reaction in a positive control and determination reference data (particularly calibration curve data) generated on the basis of the detection result are prepared and the plurality of combinations is used as teacher data. Here, in the trained model, the detection result is an explanatory variable, and the determination reference data is an objective variable.

The trained model may be, for example, a trained model generated by deep learning. For example, the trained model may be a multilayer neural network, for example, may be a deep neural network (DNN), and more specifically, may be a convolutional neural network (CNN). The multilayer neural network may include an input layer that receives an input of a detection result of a nucleic acid amplification reaction in a positive control, an output layer that outputs determination reference data, and at least one, for example, two or more intermediate layers provided between the input layer and the output layer. Furthermore, an algorithm other than deep learning may be used as the trained model. As the algorithm, for example, linear regression, multivariate adaptive regression splines (MARS), or support vector machines (SVM) may be used.

In step S116, the control unit 114 determines whether to adjust the nucleic acid amplification reaction. In the present disclosure, the control unit 114 may determine whether to adjust the nucleic acid amplification reaction on the basis of the detection result of the nucleic acid amplification reaction in the one or more positive controls and/or the determination reference data generated in step S115. An example of the determination will be described hereinafter.

### (Determination Process Based on Presence or Absence of Detection of Nucleic Acid Amplification)

In one embodiment, in a case where no nucleic acid amplification is detected in some wells with low initial template copy numbers among the one or more positive controls, the control unit 114 determines to adjust the nucleic acid amplification reaction (for example, prolong the reaction time or increase the number of reaction cycles). For example, it is determined to adjust the nucleic acid amplification reaction in a case where no nucleic acid amplification is detected in a sample having the lowest initial template copy number, in a well having the lowest copy number and a well having the second lowest copy number, or in the well having the lowest copy number and wells having the second and third lowest copy numbers among the one or more positive controls. In the control unit 114, the detection of no nucleic acid amplification may mean that the Tt value or the Ct value by the amplification reaction is not acquired after the lapse of the predetermined reaction time or after the execution of the predetermined number of reaction cycles, or that the fluorescence signal detected after the lapse of the predetermined reaction time or after the execution of the predetermined number of reaction cycles does not reach the predetermined threshold.

More appropriate calibration curve data can be obtained by adjusting the nucleic acid amplification reaction as described above.

(Determination Process with Reference to Variation Data)

In another embodiment, the control unit 114 can determine whether to adjust the nucleic acid amplification reaction on the basis of variation data regarding the Tt value or the Ct value acquired for each of the one or more positive controls. There is a case where a variation of the Tt or Ct value reflects a matrix effect. Therefore, the variation data is useful for determining whether to adjust the nucleic acid amplification reaction. The variation data includes, for example, any one, two, or three of a coefficient of variation, a standard deviation, and a variance, and particularly includes a coefficient of variation. The use of the variation data enables prolongation of the reaction time in consideration of the matrix effect.

As described in the above two determination processes, the control unit 114 may determine whether to adjust the nucleic acid amplification reaction on the basis of the Tt value or the Ct value of the nucleic acid amplification reaction in the one or more positive controls in the present disclosure.

### (Determination Process Using Standard Reference Data)

In still another embodiment of the present disclosure, the control unit 114 can determine whether to adjust the nucleic acid amplification reaction on the basis of the determination reference data generated in step S115. In this case, the control unit 114 may determine whether to adjust the nucleic acid amplification reaction on the basis of the determination reference data and standard reference data. The standard reference data is determination reference data acquired in advance, and is, for example, ideal determination reference data or average determination reference data. The standard reference data is created on the basis of a plurality of pieces of determination reference data. The control unit 114 may hold the standard reference data in advance prior to the nucleic acid analysis process, or may acquire the standard reference data from the server 120 via the network 150 while executing the nucleic acid analysis process.

In this embodiment, the control unit 114 may determine whether to adjust the nucleic acid amplification reaction on the basis of the variation data in addition to the determination reference data and the standard reference data.

### (Determination Process Using Trained Model)

In still another embodiment of the present disclosure, the control unit 114 determines whether to adjust the nucleic acid amplification reaction using a trained model. The trained model may be, for example, a trained model generated by machine learning in which at least one combination, preferably a plurality of combinations of determination reference data (particularly, calibration curve data, more specifically, a slope of a calibration curve) and variation data regarding the Tt value or the Ct value measured when the determination reference data is acquired is prepared, and the plurality of combinations is used as teacher data. Here, in the trained model, the determination reference data is an explanatory variable, and the variation data is an objective variable.

The trained model may be, for example, a trained model generated by deep learning. For example, the trained model may be a multilayer neural network, for example, may be a deep neural network (DNN), and more specifically, may be a convolutional neural network (CNN). The multilayer neural network may include an input layer that receives an input of the determination reference data, an output layer that outputs the variation data regarding the Tt value or the Ct value, and at least one, for example, two or more intermediate layers provided between the input layer and the output layer. Furthermore, an algorithm other than deep learning may be used as the trained model. As the algorithm, for example, linear regression, multivariate adaptive regression splines (MARS), or support vector machines (SVM) may be used.

In a case where the control unit 114 determines to adjust the nucleic acid amplification reaction in step S116 described above, the process proceeds to step S117. In a case where the control unit 114 determines not to adjust the nucleic acid amplification reaction in step S116, the process proceeds to step S118.

In step S117, the control unit 114 controls the amplification reaction execution unit 111 to adjust the nucleic acid amplification reaction. In a case where the nucleic acid amplification reaction is an isothermal nucleic acid amplification reaction, the control unit 114 prolongs or shortens the time for the nucleic acid amplification reaction by the amplification reaction execution unit 111, that is, the time for maintaining the temperature at which amplification is performed. In a case where the nucleic acid amplification reaction is PCR, the control unit 114 increases or decreases the number of PCR cycles by the amplification reaction execution unit 111. In this manner, the control unit 115 may adjust the reaction time or the number of reaction cycles of the nucleic acid amplification reaction in the nucleic acid amplification reaction adjustment process.

In step S117, the nucleic acid amplification reaction is adjusted, and the detection unit 113 further acquires a detection result of the nucleic acid amplification reaction.

After step S117, the control unit 114 returns the process to step S112, and acquires the detection result acquired by the detection unit 113 after the adjustment. Then, the control unit 114 executes steps S112 to S116 on the basis of the detection result acquired before the adjustment and the detection result acquired after the adjustment. Note that the invalidity determination process in step S113 may be omitted after the adjustment, that is, the control unit 114 may cause the process to proceed to step S115 after completion of step S112.

In step S118, the control unit 114 ends the adjustment process in step S102, and then, causes the process to proceed to the determination process in step S103. In this manner, in the present disclosure, the control unit 114 may execute a determination result generation process of generating a determination result for the biological sample in response to determining not to adjust the nucleic acid amplification reaction. Since the determination result is generated after confirming that the nucleic acid amplification reaction adjustment process is unnecessary in this manner, a more appropriate determination result can be generated.

### (3-2) Determination Process

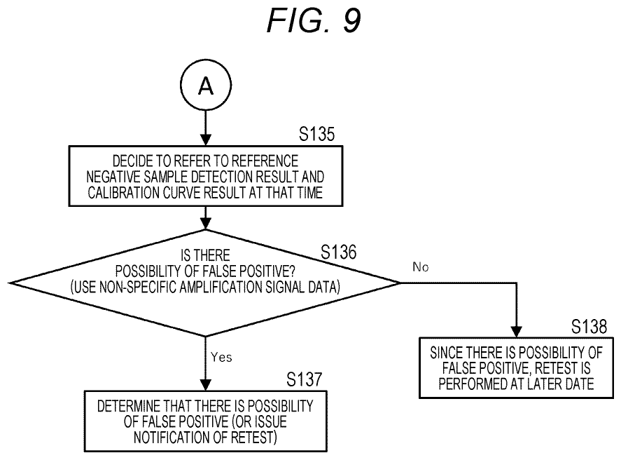

FIGS. 8 and 9 illustrate an example of a flowchart of a process executed by the control unit 114 in step S103.

In step S121, the control unit 114 starts a determination process regarding the presence and/or amount of the target nucleic acid in the biological sample.

In step S122, the control unit 114 determines whether the target nucleic acid is detected in the nucleic acid amplification reaction in the biological sample. For the determination, the control unit 114 acquires a detection result of the nucleic acid amplification reaction in a well containing the biological sample. The control unit 114 determines whether the target nucleic acid is detected in the nucleic acid amplification reaction in the biological sample on the basis of the acquired detection result.

For example, the control unit 114 may determine that the target nucleic acid is detected in the nucleic acid amplification reaction in the biological sample in a case where a fluorescence signal (or an average value of fluorescence signals over a predetermined period) at a predetermined time point after the start of the amplification reaction out of the acquired detection result is equal to or more than a predetermined threshold, and determine that the target nucleic acid is not detected in the nucleic acid amplification reaction in the biological sample in a case where the fluorescence signal is less than the predetermined threshold.

In step S122, the control unit 114 may identify the amount of the target nucleic acid in the biological sample (the amount of the target nucleic acid contained in the biological sample before the nucleic acid amplification reaction is executed, which is also referred to as a “target nucleic acid amount in a sample”) on the basis of the acquired detection result and the determination reference data generated in step S102 (the last generated determination reference data in a case where the determination reference data is generated a plurality of times in step S102). Then, whether the target nucleic acid is detected may be determined on the basis of the identified amount.

For example, the control unit 114 may determine that the target nucleic acid is detected in the nucleic acid amplification reaction in the biological sample in a case where the target nucleic acid amount in the sample is equal to or more than a predetermined threshold, and may determine that the target nucleic acid is not detected in the nucleic acid amplification reaction in the biological sample in a case where the target nucleic acid amount in the sample is less than the predetermined threshold.

In this manner, the control unit 114 may be configured to execute the wrong answer determination process to be described later in a case where the detection result of the nucleic acid amplification reaction in the biological sample does not satisfy a predetermined condition in step S122.

For example, the control unit 114 may determine whether the target nucleic acid is present in the biological sample on the basis of the fluorescence signal in the well containing the biological sample and the calibration curve data included in the determination reference data. Furthermore, the control unit 114 may identify the target nucleic acid amount in the sample (particularly, a copy number of the target nucleic acid in the biological sample before the nucleic acid amplification reaction) on the basis of the fluorescence signal and the calibration curve data.

In a case where it is determined in step S122 that the target nucleic acid is not present in the biological sample, the control unit 114 causes the process to proceed to step S123.

In a case where it is determined in step S122 that the target nucleic acid is present in the biological sample, the control unit 114 causes the process to proceed to step S124.

In step S123, the control unit 114 generates a determination result indicating that the biological sample does not contain the target nucleic acid, that is, the biological sample is negative. After generating the determination result, the control unit 114 causes the process to proceed to step S104.

In step S124, the control unit 114 determines whether there is a possibility that the determination result in step S122 is a wrong answer. In the present specification, a process of executing this determination is also referred to as the “wrong answer determination process”. step S123 is a step executed in a case where it is determined in step S122 that the target nucleic acid is present in the biological sample (that is, it can also be said as a case where it is determined to be positive), and thus, can also be said to be a step of determining whether the determination result is a false positive. With this determination, a possibility of a false positive can be reduced, and determination accuracy can be improved. Hereinafter, an example of the wrong answer determination process will be described.

### (Embodiment of Using Threshold)

In one embodiment of the present disclosure, in step S124, the control unit 114 refers to the target nucleic acid amount in the sample identified in step S122. Then, for example, the control unit 114 determines whether the target nucleic acid amount in the sample is equal to or less than a predetermined threshold.

In a case where the target nucleic acid amount in the sample is equal to or more than the predetermined threshold (or more than the threshold), the control unit 114 determines that there is no possibility of a wrong answer (that is, that it is a true positive), and causes the process to proceed to step S125.

In a case where the target nucleic acid amount in the sample is less than the predetermined threshold (or equal to or less than the threshold), the control unit 114 determines that there is a possibility of a wrong answer (that is, there is a possibility of a false positive) and causes the process to proceed to step S126.

The predetermined threshold may be, for example, a value equal to or less than a copy number in a well having the lowest initial template copy number among the one or more positive controls, or may be a value equal to or less than a copy number in a well having the second lowest initial template copy number. In a case where the amount of the target nucleic acid contained in the biological sample is less than such a threshold (equal to or less than the threshold), there is a possibility that a fluorescence signal generated in the nucleic acid amplification for the biological sample is not caused by the target nucleic acid, but is a non-specific amplification signal (for example, a signal derived from a primer dimer or a probe dimer). Therefore, the determination on the basis of such a threshold is useful for improving test accuracy, and it is possible to deal with a fluctuation in the determination in the case of a low copy number.

### (Embodiment of Using Threshold and Reference Negative Detection Result)

In “Embodiment of using threshold” as described above, the control unit 114 determines that there is a possibility of a wrong answer in a case where the target nucleic acid amount in the sample is less than the predetermined threshold (or equal to or less than the threshold).

In another embodiment of the present disclosure, in a case where the target nucleic acid amount in the sample is less than the predetermined threshold (or equal to or less than the threshold), the control unit 114 refers to a detection result of a nucleic acid amplification reaction in a negative sample (also referred to as a “reference negative detection result” in the present specification) and determines whether there is a possibility of a wrong answer. The reference negative detection result may mean a fluorescence signal detection result determined to be negative.

The nucleic acid amplification reaction in the negative sample may be another nucleic acid amplification reaction other than the nucleic acid amplification reaction executed on the biological sample, and more specifically, may be another nucleic acid amplification reaction that has been already executed before the nucleic acid amplification reaction executed on the biological sample is started.

For example, the control unit 114 may determine whether there is a possibility of a wrong answer on the basis of the detection result of the nucleic acid amplification reaction in the well containing the biological sample and the reference negative detection result. As an example, the control unit 114 may determine that there is a possibility of a wrong answer in a case where the former detection result is similar to the latter detection result, and the control unit 114 may determine that there is no possibility of a wrong answer in a case where the former detection result is not similar to the latter detection result.

As described above, there is a case where the fluorescence signal that is the basis for a positive determination result in step S122 is caused by a non-specific amplification reaction. In this regard, it is possible to confirm whether the fluorescence signal is derived from the amplification of the target nucleic acid by referring to the reference negative detection result. Therefore, it is possible to cope with the fluctuation in the determination in the case of the low copy number.

Note that the control unit 114 may acquire the reference negative detection result from the server 120 via the network 150.

As described above, the control unit 114 may refer to the detection result of the nucleic acid amplification reaction in the negative sample in the wrong answer determination process in the present disclosure.

### (Embodiment of Using Trained Model)

In “Embodiment of using threshold and reference negative detection result” as described above, the control unit 114 refers to the detection result of the nucleic acid amplification reaction in the negative sample and determines whether there is a possibility of a wrong answer in a case where the target nucleic acid amount in the sample is less than the predetermined threshold (or equal to or less than the threshold).

In still another embodiment of the present disclosure, in step S124, the control unit 114 may determine whether there is a possibility of a wrong answer using a trained model. An example of the trained model will be described hereinafter.

An example of the trained model is, for example, a trained model generated by machine learning in which at least one data set, preferably a plurality of data sets is prepared, the data set including calibration curve data (particularly, a slope) and a Tt value or a Ct value acquired in a nucleic acid amplification reaction for a sample in which the target nucleic acid amount in the sample is less than the predetermined threshold (or equal to or less than the threshold) as explanatory variables and negative or positive determination result data for the sample as an objective variable, and the data set is used as teacher data.

Another example of the trained model may be, for example, a trained model generated by machine learning in which at least one combination, preferably a plurality of, combinations of a detection result (for example, fluorescence signal data) in a nucleic acid amplification reaction for a sample in which the target nucleic acid amount in the sample is less than the predetermined threshold (or equal to or less than the threshold) and negative or positive determination result data for the sample is prepared and the combination is used as teacher data. Here, in the trained model, the detection result is an explanatory variable, and the determination result data is an objective variable.

The trained model may be, for example, a trained model generated by deep learning. For example, the trained model may be a multilayer neural network, for example, may be a deep neural network (DNN), and more specifically, may be a convolutional neural network (CNN). The multilayer neural network may include an input layer that receives an input of the determination reference data, an output layer that outputs the variation data regarding the Tt value or the Ct value, and at least one, for example, two or more intermediate layers provided between the input layer and the output layer. Furthermore, an algorithm other than deep learning may be used as the trained model. As the algorithm, for example, linear regression, multivariate adaptive regression splines (MARS), or support vector machines (SVM) may be used.

The trained model as described above is useful for determining the possibility of a wrong answer.

In step S125, the control unit 114 generates a final determination result indicating a positive. After generating the determination result in step S125, the control unit 114 causes the process to proceed to step S104.

In step S126, the control unit 114 outputs notification data regarding a false positive on the basis of the determination result in the wrong answer determination process. The control unit 114 causes the output device to output a notification that there is a possibility of a false positive. For example, the control unit 114 causes the display device to display the notification. Therefore, the user of the system according to the present disclosure can know the possibility of the false positive in the determination result based on the nucleic acid amplification reaction.

The notification output in step S126 may include, for example, a screen inquiring whether to perform additional analysis. In this manner, the control unit 114 may output the screen regarding whether to execute the additional analysis for generating a determination result for the biological sample on the basis of the determination result in the wrong answer determination process.

In step S127, the control unit 114 determines whether to perform the additional analysis.

For example, the control unit 114 determines to perform the additional analysis in response to the user selecting to perform the additional analysis via the screen output in step S126. In a case where it is determined to perform the additional analysis, the control unit 114 causes the process to proceed to step S128.

On the other hand, the control unit 114 determines not to perform the additional analysis in response to the user selecting not to perform the additional analysis via the screen output in step S126. In a case where it is determined not to perform the additional analysis, the control unit 114 causes the process to proceed to step S134.

In step S134, the control unit 114 determines that there is a possibility of a false positive and causes the process to proceed to S135.

In step S128, the control unit 114 starts the additional analysis. The additional analysis is analysis for confirming whether an amplification product obtained by the nucleic acid amplification reaction for the biological sample is an amplification product of the target nucleic acid. The additional analysis is, for example, melting curve analysis, but is not limited thereto. The melting curve analysis may be performed as described in Patent Document 3, for example. In order to perform the melting curve analysis, the control unit 114 causes the amplification reaction execution unit 111 to control the temperature of the biological sample as will be described below. Furthermore, the control unit 114 causes the detection unit 113 to detect fluorescence generated by the temperature control, and obtains data (for example, plot data) regarding a change in fluorescence intensity with respect to a change in temperature. The control unit 114 executes the melting curve analysis on the basis of the data to determine whether the amplification product obtained by the nucleic acid amplification reaction is the amplification product of the target nucleic acid.

For example, in step S128, in order to perform the melting curve analysis, a double strand formed by hybridization of an amplification product and a probe nucleic acid chain for melting curve analysis is formed in a well of the biological sample. In order to form the double strand, the biological sample is cooled to a temperature lower than a melting temperature of the probe nucleic acid chain. When the temperature of the biological sample becomes lower than the melting temperature of the probe nucleic acid chain by the cooling, the probe nucleic acid chain hybridizes to the amplification product to form the double strand. Typically, cooling is performed from a reaction temperature (for example, around 65° C.) of a nucleic acid amplification reaction to room temperature to 40° C. For the cooling, for example, the amplification reaction execution unit 111 regulates the temperature of the biological sample. Note that the probe nucleic acid chain may be contained in a well in advance before the nucleic acid analysis process according to the present disclosure is started. The probe nucleic acid chain may be appropriately designed by those skilled in the art. The probe nucleic acid chain may be designed to have a predetermined melting temperature, for example, as described in Patent Document 3. Furthermore, the probe nucleic acid chain may be given a predetermined label (fluorescent label) so as to generate fluorescence or eliminate fluorescence by the formation of the double strand.

Before the cooling is performed, the amplification product (amplified nucleic acid chain) may be thermally denatured. The thermal denaturation is usually carried out by heating to 90 to 100° C., preferably around 95° C. The thermal denaturation partially dissociates a double-strand forming portion of the amplification product. Then, the probe nucleic acid chain hybridizes to such a dissociated portion in the cooling.

After the double strand is formed, the temperature of the biological sample is raised. In order to raise the temperature, for example, the amplification reaction execution unit 111 regulates the temperature of the biological sample. Due to the temperature rise, the double strand is melted to form a single-stranded nucleic acid. The temperature may be raised, for example, from the temperature after the cooling to around 90° C. During the temperature rise, the detection unit 113 detects the fluorescence intensity from the biological sample. Therefore, data (for example, plot data) regarding a change in the fluorescence intensity with respect to a temperature change is obtained. The control unit 114 executes the melting curve analysis on the basis of the data to determine whether the amplification product obtained by the nucleic acid amplification reaction is the amplification product of the target nucleic acid.

In step S129, the control unit 114 determines whether the target nucleic acid is contained in the biological sample on the basis of a result of the additional analysis.

In a case where it is determined that the target nucleic acid is contained, the control unit 114 determines that it is positive and causes the process to proceed to step S130.

In a case where it is determined that the target nucleic acid is not contained, the control unit 114 determines that it is not positive and causes the process to proceed to step S131.

In step S130, the control unit 114 generates a determination result indicating a positive. After generating the determination result, the control unit 114 causes the process to proceed to step S104.

In step S131, the control unit 114 determines whether it can be determined to be negative on the basis of the result of the additional analysis. With this step, it is possible to prevent a wrong answer determination from being made in a case where it is difficult to accurately make a positive or negative determination even by the additional analysis.

In a case where it can be determined to be negative, the control unit 114 causes the process to proceed to step S133.

In a case where it cannot be determined to be negative, the control unit 114 causes the process to proceed to step S134.

In step S132, the control unit 114 generates a final determination result indicating a negative. After generating the determination result, the control unit 114 causes the process to proceed to step S104.

In step S133, the control unit 114 decides that the determination by the additional analysis is difficult, and causes the process to proceed to step S135.

In step S135, the control unit 114 decides to execute a determination process using a reference negative detection result (negative control data) and calibration curve data created when the reference negative detection result has been obtained, and causes the process to proceed to step S136.

In step S136, the control unit 114 determines whether there is a possibility of a false positive with reference to the above-described reference negative detection result and calibration curve data. In a case where it is determined that there is a possibility of a false positive, the control unit 114 causes the process to proceed to step S137. In a case where it is determined that there is no possibility of a false positive, the control unit 114 causes the process to proceed to step S138.

For example, the control unit 114 may compare the reference negative detection result with the detection result in the biological sample, and determine that there is no possibility of a false positive in a case where these detection results are similar. Furthermore, in a case where these detection results are not similar, it may be determined that there is a possibility of a false positive.

In step S138, the control unit 114 determines to perform a retest at a later date or recommend the retest since there is a possibility of a false negative.

In step S137, the control unit 114 determines that there is a possibility of a false positive (or issues a notification to perform the retest).

### (3-3) Modified Example of Determination Process

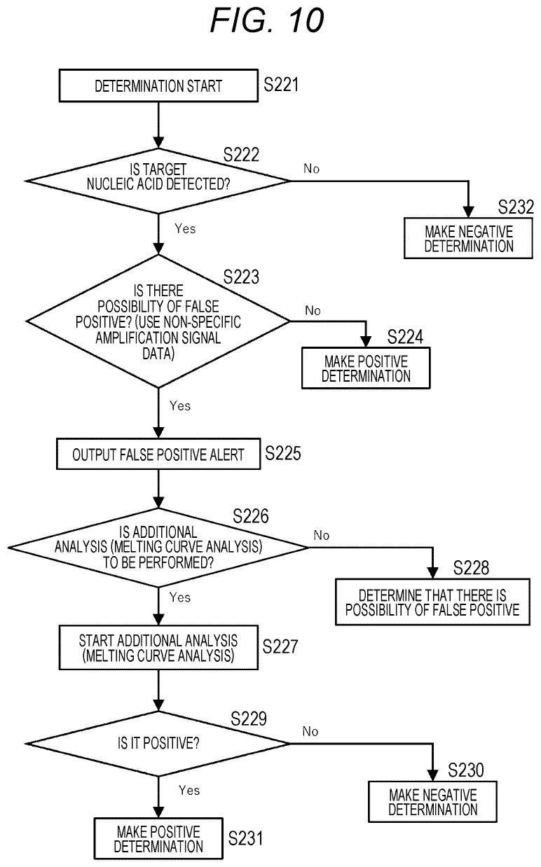

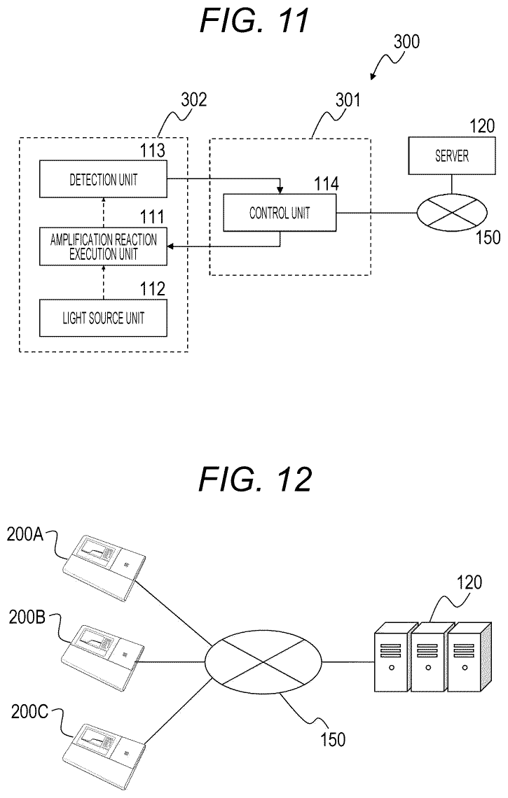

FIGS. 10 and 11 illustrate a modified example of a processing flow executed by the control unit 114 in step S103.

In step S221, the control unit 114 starts a determination process regarding the presence and/or amount of the target nucleic acid in the biological sample.

Step S222 is the same as step S122 described in (3-2) above, and the description of step S122 also applies to step S222.

In a case where it is determined in step S222 that the target nucleic acid is present in the biological sample, the control unit 114 causes the process to proceed to step S223. This case is also referred to as a “case where it is determined to be positive in the determination of step S222” in the description of this flowchart.

Furthermore, in a case where it is determined in step S222 that the target nucleic acid is not present in the biological sample, the control unit 114 causes the process to proceed to step S232. This case is also referred to as a “case where it is determined to be negative in the determination of step S222” in the description of this flowchart.

FIG. 10 illustrates an example of the flowchart of the process executed by the control unit 114 in the case where it is determined to be positive in the determination of step S222. On the other hand, FIG. 11 illustrates an example of a flowchart of a process executed by the control unit 114 in the case where it is determined to be negative in the determination of step S222. Hereinafter, the example of the processing flow in the case where it is determined to be positive in the determination of step S222 will be described first, and the example of the processing flow in the case where it is determined to be negative in the same determination will be described next.

(In Case where it is Determined to be Positive in Step S222)

In step S223, the control unit 114 executes a wrong answer determination process of determining whether there is a possibility that the determination result in step S222 is a wrong answer. The wrong answer determination process is a step executed in a case where it is determined in step S222 that the target nucleic acid is present in the biological sample (that is, it can also be said as the case where it is determined to be positive), and thus, can also be said to be a step of determining whether the determination result is a false positive. With this determination, a possibility of a false positive can be reduced, and determination accuracy can be improved.

Step S223 is the same as step S124 described in (3-2) above, and the description of step S124 also applies to step S223.

In step S224, the control unit 114 generates a final determination result indicating a positive. After generating the determination result in step S125, the control unit 114 causes the process to proceed to step S104.

In step S225, the control unit 114 may cause the output device to output a notification that there is a possibility of a false positive. Therefore, the user of the system according to the present disclosure can know the possibility of the false positive in the determination result based on the nucleic acid amplification reaction.

The notification output in step S225 may include, for example, a screen inquiring whether to perform additional analysis.

In step S226, the control unit 114 determines whether to perform the additional analysis.

For example, the control unit 114 determines to perform the additional analysis in response to the user selecting to perform the additional analysis via the screen output in step S225. In a case where it is determined to perform the additional analysis, the control unit 114 causes the process to proceed to step S227.

On the other hand, the control unit 114 determines not to perform the additional analysis in response to the user selecting not to perform the additional analysis via the screen output in step S225. In a case where it is determined not to perform the additional analysis, the control unit 114 causes the process to proceed to step S228.

In step S227, the control unit 114 starts the additional analysis. Step S227 is the same as step S128 described in (3-2) above, and the description of step S128 also applies to step S227.

In step S228, the control unit 114 generates a determination result indicating that there is a possibility of a false positive. After generating the determination result, the control unit 114 causes the process to proceed to step S104.

The determination result may include a notification for prompting execution of a nucleic acid amplification test again, or may include a notification for prompting execution of another test.

Through the notification, a subject can know that there is a possibility of a false positive. Therefore, it is possible to prompt the subject to be tested again.

In step S229, the control unit 114 determines whether the target nucleic acid is contained in the biological sample on the basis of a result of the additional analysis.

In a case where it is determined that the target nucleic acid is contained, the control unit 114 determines that it is positive and causes the process to proceed to step S231.

In a case where it is determined that the target nucleic acid is not contained, the control unit 114 determines that it is negative and causes the process to proceed to step S230.

In step S230, the control unit 114 generates a final determination result indicating a negative. After generating the determination result, the control unit 114 causes the process to proceed to step S104.

In step S231, the control unit 114 generates a final determination result indicating a positive. After generating the determination result, the control unit 114 causes the process to proceed to step S104.

(In Case where it is Determined to be Negative in Determination of Step S222)

In a case where it is determined in step S222 that the target nucleic acid is not present, the control unit causes the process to proceed to step S232. In step S232, the control unit 114 generates a final determination result indicating a negative. After generating the determination result, the control unit 114 causes the process to proceed to step S104.

### (4) First Modified Example of Nucleic Acid Analysis System

FIG. 11 illustrates a modified example of a nucleic acid analysis system according to the present disclosure. A nucleic acid analysis system 300 illustrated in the drawing includes an information processing device 301, and the information processing device includes the control unit 114 described above. That is, the nucleic acid analysis system according to the present disclosure may be configured to include only the control unit 114 described above. In this case, the amplification reaction execution unit 111, the light source unit 112, and the detection unit 113 may be included in a nucleic acid amplification device 302 different from the information processing device 301 as illustrated in the drawing.

### (5) Second Modified Example of Nucleic Acid Analysis System

FIG. 12 illustrates another modified example of a nucleic acid analysis system according to the present disclosure. In the nucleic acid analysis system illustrated in the drawing, a plurality of nucleic acid amplification devices 200 (indicated as 200A to 200C in the drawing) is connected to the server 120 via the network 150. Note that the number of the nucleic acid amplification devices 200 connected to the server 120 is not limited to that illustrated in FIG. 12, and a large number of nucleic acid amplification devices may be connected. Therefore, each of the nucleic acid amplification devices may be configured to be capable of transmitting various types of acquired data (for example, a detection result and a determination result) to the server 120 via the network 150. Therefore, a large number of pieces of data can be accumulated in the server 120. The contents described with reference to FIG. 3 apply to a specific configuration of the server 120.

For example, the nucleic acid amplification device 200 can be configured to transmit one or more types of data generated in the processes executed by the nucleic acid analysis system described in (3) to the server 120. The transmitted data, for example, may be one or more of detection result data of a nucleic acid amplification reaction acquired in step S112 described above; a determination result in step S113; determination reference data generated in step S115; a determination result in step S116 and data (for example, the presence or absence of detection of nucleic acid amplification, variation data, and determination reference data) as the basis of the determination result; a detection result acquired in step S122 and a determination result based on the detection result (and determination reference data referred to for the determination); determination results in steps S124, S136, S223, and S232 and data as the basis of the determination; and data obtained by additional analysis in steps S128, S227, and S237 and a determination result based on the data, or may be all of them. Each piece of the data may be transmitted to the server 120 in association with sample identification data (for example, a sample ID) for identifying a biological sample or together with the sample identification data.

Furthermore, the server 120 may generate any one or more of the trained models described above using pieces of the accumulated data.

Furthermore, the server 120 may hold detection results of nucleic acid amplification reactions in negative samples. The server 120 may be configured to be capable of accumulating the detection results of the nucleic acid amplification reactions in the negative samples transmitted from the control unit 114. The trained model described above may be generated using the accumulated detection results.

Furthermore, the server 120 may hold standard reference data. The server may generate or update the standard reference data on the basis of pieces of the determination reference data accumulated as described above.

Furthermore, the control unit 114 has been described to be included in the nucleic acid amplification device in (3) described above, but the control unit 114 may be included in the server 120. That is, the server 120 may execute the information processing performed by the control unit 114 described in (3) above. That is, the nucleic acid analysis system of the present disclosure may include a nucleic acid amplification device including the amplification reaction execution unit 111 and a server device including the control unit 114.

Note that the present disclosure can also have the following configurations.

[1]

A nucleic acid analysis system including:
an amplification reaction execution unit that executes a nucleic acid amplification reaction on a biological sample; and
a control unit that controls the amplification reaction execution unit,
in which the control unit is configured to
determine whether to adjust a nucleic acid amplification reaction on the basis of a detection result of the nucleic acid amplification reaction in one or more positive controls, and
generate a determination result for the biological sample on the basis of the detection result of the nucleic acid amplification reaction in the one or more positive controls and a detection result of the nucleic acid amplification reaction in the biological sample.
   [2]

The nucleic acid analysis system according to [1], in which
the control unit
generates determination reference data on the basis of the detection result of the nucleic acid amplification reaction in the one or more positive controls, and then,
determines whether to adjust the nucleic acid amplification reaction on the basis of the determination reference data.
   [3]

The nucleic acid analysis system according to [2], in which the control unit determines whether to adjust the nucleic acid amplification reaction on the basis of the determination reference data and standard reference data.

[4]

The nucleic acid analysis system according to [2] or [3], in which the control unit determines whether to adjust the nucleic acid amplification reaction on the basis of a Tt value or a Ct value of the nucleic acid amplification reaction in the one or more positive controls.

[5]

The nucleic acid analysis system according to any one of [2] to [4], in which the control unit determines whether to adjust the nucleic acid amplification reaction using a trained model.

[6]

The nucleic acid analysis system according to any one of [1] to [5], in which the control unit adjusts a reaction time or the number of reaction cycles of the nucleic acid amplification reaction in adjustment of the nucleic acid amplification reaction.

[7]

The nucleic acid analysis system according to any one of [2] to [6], in which the control unit determines that the nucleic acid amplification reaction for the biological sample is invalid in a case where the determination reference data cannot be generated.

[8]

The nucleic acid analysis system according to any one of [1] to [7], in which the control unit executes a determination result generation process of generating a determination result for the biological sample in response to determining not to adjust the nucleic acid amplification reaction.

[9]

The nucleic acid analysis system according to [8], in which the control unit determines whether a target nucleic acid is detected in the biological sample in the determination result generation process.

[10]

The nucleic acid analysis system according to [9] or [10], in which the control unit further executes a wrong answer determination process of determining whether a determination result regarding whether the target nucleic acid has been detected in the biological sample is a wrong answer in the determination result generation process.

[11]

The nucleic acid analysis system according to [10], in which the control unit executes the wrong answer determination process in a case where the detection result of the nucleic acid amplification reaction in the biological sample does not satisfy a predetermined condition.

[12]

The nucleic acid analysis system according to [11], in which the control unit refers to a detection result of the nucleic acid amplification reaction in a negative sample in the wrong answer determination process.

[13]

The nucleic acid analysis system according to [12], in which the detection result of the nucleic acid amplification reaction in the negative sample includes a detection result of the nucleic acid amplification reaction executed on another sample determined to be negative.

[14]

The nucleic acid analysis system according to any one of [10] to [13], in which the control unit executes the wrong answer determination process using a trained model.

[15]

The nucleic acid analysis system according to any one of [10] to [14], in which the control unit outputs notification data regarding a false positive on the basis of a determination result in the wrong answer determination process.

[16]

The nucleic acid analysis system according to any one of [10] to [15], in which the control unit outputs a screen regarding whether to execute additional analysis for generating a determination result for the biological sample on the basis of the determination result in the wrong answer determination process.

[17]

The nucleic acid analysis system according to [16], in which the additional analysis is melting curve analysis.

[18]

The nucleic acid analysis system according to any one of [1] to [17], further including a nucleic acid amplification device including the amplification reaction execution unit and the control unit.

[19]

The nucleic acid analysis system according to any one of [1] to [17], further including:
a nucleic acid amplification device including the amplification reaction execution unit; and
an information processing device including the control unit.
   [20]

The nucleic acid analysis system according to any one of [1] to [17], further including:
a nucleic acid amplification device including the amplification reaction execution unit; and
a server device including the control unit.
   [21]

A nucleic acid analysis system including
the control unit being configured to
determine whether to adjust the nucleic acid amplification reaction on the basis of a detection result of a nucleic acid amplification reaction in one or more positive controls, and
generate a determination result for the biological sample on the basis of the detection result of the nucleic acid amplification reaction in the one or more positive controls and a detection result of the nucleic acid amplification reaction in the biological sample.

### REFERENCE SIGNS LIST

100 Nucleic acid analysis system
111 Amplification reaction execution unit
112 Light source unit
113 Detection unit
114 Control unit

1. A nucleic acid analysis system comprising:
an amplification reaction execution unit that executes a nucleic acid amplification reaction on a biological sample; and
a control unit that controls the amplification reaction execution unit,
wherein the control unit is configured to
determine whether to adjust a nucleic acid amplification reaction on a basis of a detection result of the nucleic acid amplification reaction in one or more positive controls, and
generate a determination result for the biological sample on a basis of the detection result of the nucleic acid amplification reaction in the one or more positive controls and a detection result of the nucleic acid amplification reaction in the biological sample.

2. The nucleic acid analysis system according to claim 1, wherein
the control unit
generates determination reference data on a basis of the detection result of the nucleic acid amplification reaction in the one or more positive controls, and then,
determines whether to adjust the nucleic acid amplification reaction on a basis of the determination reference data.

3. The nucleic acid analysis system according to claim 2, wherein the control unit determines whether to adjust the nucleic acid amplification reaction on a basis of the determination reference data and standard reference data.

4. The nucleic acid analysis system according to claim 2, wherein the control unit determines whether to adjust the nucleic acid amplification reaction on a basis of a Tt value or a Ct value of the nucleic acid amplification reaction in the one or more positive controls.

5. The nucleic acid analysis system according to claim 2, wherein the control unit determines whether to adjust the nucleic acid amplification reaction using a trained model.

6. The nucleic acid analysis system according to claim 1, wherein the control unit adjusts a reaction time or a number of reaction cycles of the nucleic acid amplification reaction in adjustment of the nucleic acid amplification reaction.

7. The nucleic acid analysis system according to claim 2, wherein the control unit determines that the nucleic acid amplification reaction for the biological sample is invalid in a case where the determination reference data is not be generatable.

8. The nucleic acid analysis system according to claim 1, wherein the control unit executes a determination result generation process of generating a determination result for the biological sample in response to determining not to adjust the nucleic acid amplification reaction.

9. The nucleic acid analysis system according to claim 8, wherein, in the determination result generation process, the control unit determines whether a target nucleic acid is detected in the biological sample.

10. The nucleic acid analysis system according to claim 9, wherein, in the determination result generation process, the control unit further executes a wrong answer determination process of determining whether a determination result regarding whether the target nucleic acid has been detected in the biological sample is a wrong answer.

11. The nucleic acid analysis system according to claim 10, wherein the control unit executes the wrong answer determination process in a case where the detection result of the nucleic acid amplification reaction in the biological sample does not satisfy a predetermined condition.

12. The nucleic acid analysis system according to claim 11, wherein the control unit refers to a detection result of the nucleic acid amplification reaction in a negative sample in the wrong answer determination process.

13. The nucleic acid analysis system according to claim 12, wherein the detection result of the nucleic acid amplification reaction in the negative sample includes a detection result of the nucleic acid amplification reaction executed on another sample determined to be negative.

14. The nucleic acid analysis system according to claim 10, wherein the control unit executes the wrong answer determination process using a trained model.

15. The nucleic acid analysis system according to claim 10, wherein the control unit outputs notification data regarding a false positive on a basis of a determination result in the wrong answer determination process.

16. The nucleic acid analysis system according to claim 10, wherein the control unit outputs a screen regarding whether to execute additional analysis for generating a determination result for the biological sample on a basis of the determination result in the wrong answer determination process.

17. The nucleic acid analysis system according to claim 16, wherein the additional analysis is melting curve analysis.

18. The nucleic acid analysis system according to claim 1, further comprising:
a nucleic acid amplification device including the amplification reaction execution unit; and
an information processing device including the control unit.

19. The nucleic acid analysis system according to claim 1, further comprising:
a nucleic acid amplification device including the amplification reaction execution unit; and
a server device including the control unit.

20. A nucleic acid analysis system comprising
a control unit that controls a nucleic acid amplification reaction for a biological sample,
wherein the control unit is configured to
determine whether to adjust the nucleic acid amplification reaction on a basis of a detection result of a nucleic acid amplification reaction in one or more positive controls, and
generate a determination result for the biological sample on a basis of the detection result of the nucleic acid amplification reaction in the one or more positive controls and a detection result of the nucleic acid amplification reaction in the biological sample.
